(19) United States
(12) Patent Application Publication
(10) Pub. No.: US 2023/0233424 A1
(43) Pub. Date: Jul. 27, 2023

(54) USE OF A MINERAL BLEND AS COSMETIC AGENT FOR WET COSMETIC COMPOSITIONS

(71) Applicant: Omya International AG, Oftringen (CH)

(72) Inventors: Lalit SHARMA, Zofingen (CH); Tanja BUDDE, Brittnau (CH); Stefan LANDER, Rupperswil (CH)

(73) Assignee: Omya International AG, Oftringen (CH)

(21) Appl. No.: 17/998,293

(22) PCT Filed: May 19, 2021
(86) PCT No.: PCT/EP2021/063347

§ 371 Date: Nov. 9, 2022

(30) Foreign Application Priority Data
May 29, 2020 (EP) 20177424.7

(51) Int. Cl. A61K 8/19; A61K 8/02; A61Q 1/12

(52) U.S. Cl. CPC A61K 8/19; A61K 8/0241; A61Q 1/12; A61K 2800/30; A61K 2800/412; A61K 2800/651; A61K 2800/621

(57) ABSTRACT

The present invention relates to the use of a mineral blend as cosmetic agent for a wet cosmetic composition, wherein the mineral blend comprises a first component being selected from natural ground calcium carbonate and/or precipitated calcium carbonate, and a second component being a surface-reacted calcium carbonate, as well as to a wet cosmetic composition comprising said mineral blend.

The present invention relates to the field of cosmetic compositions, and in particular to the use of a mineral blend as cosmetic agent for wet cosmetic compositions, and a wet cosmetic composition comprising said mineral blend.

Wet cosmetic compositions are used for a variety of different applications. For example, a wet cosmetic compositions may be applied for cleaning, nurturing, or rehydrating skin or hair. Wet cosmetic compositions may also be used to colour skin or hair, to hide blemishes, conceal or diminish fine lines or wrinkles, minimize pores or to change and/or even out the skin tone. Furthermore, wet cosmetic compositions are used to modify the skin feel. For example, the absorption of water, moisture, lipids or essential oils from the wet cosmetic composition into the skin surface can provide a smoothened skin having a softer skin feel and/or causing less friction with e.g. clothing, diapers, gloves and/or mechanic or electronic shavers. Besides providing one or more of the aforementioned effects, a wet cosmetic composition usually has to fulfil further criteria to be fully accepted by the customer. For example, the wet cosmetic composition has to be fast absorbing, should deliver hydration, needs to be non-greasy as well as non-tacky, must be easily applicable by the user and/or should provide the user with a pleasant feeling during application. Moreover, especially in case of deodorants, customers expect a dry and comfortable skin feeling during and/or after application and a long-lasting scent and feeling of freshness.

Furthermore, the wet cosmetic composition should be free of any components, which may be considered a health concern for the user. For example, certain silicates such as talc or talc-containing material are believed to increase the risk of lung conditions, if accidentally inhaled due to wrong use of the composition and/or use thereof in excess. In case of wet cosmetic compositions this is a rather low risk. However, if a make-up product, for example, containing talc gets into the eyes, it can cause redness and the sensation that something is stuck in the eye. Moreover, there is increasing evidence that talc can cause problems such as infections or inflammations if it gets under the skin through an open wound, for example, a sore skin or a small cut obtained from shaving. In view thereof, customers usually prefer wet cosmetic composition, which are free of components such as talc or talc-containing material.

In view of the foregoing, there is a continuing need in the art for cosmetic agents for wet cosmetic compositions, and especially cosmetic agents which may be used as a replacement for components being considered a health concern such as talc-containing materials.

Accordingly, it is an object of the present invention to provide a cosmetic agent, which may improve the sensory properties of a wet cosmetic composition, for example, the skin appearance, skin feel, skin smoothness and/or the applicability of the wet cosmetic composition. It would be desirable that the cosmetic agent can replace talc or talc-containing material. It would also be desirable that the cosmetic agent is obtained from natural sources and is suitable for organic cosmetics.

It is also an object of the present invention to provide a wet cosmetic composition having improved sensory properties. It is desirable that the wet cosmetic composition can be easily applied to the skin and forms an even and uniform film on the skin. Moreover, it would be desirable that the wet cosmetic composition is less greasy and less sticky. It would also be desirable that the cosmetic composition exhibits a good spreadability and dries fast. In particular, it would be desirable to provide a wet cosmetic composition, especially a baby cream or diaper rush cream, which is free of talc or talc-containing material.

The foregoing and other objects are solved by the subject-matter defined in the independent claims.

According to one aspect of the present invention, use of a mineral blend as cosmetic agent for a wet cosmetic composition is provided, wherein the mineral blend comprises

a first component being selected from natural ground calcium carbonate and/or precipitated calcium carbonate, and

a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source.

According to another aspect of the present invention, a wet cosmetic composition comprising a mineral blend is provided, wherein the mineral blend comprises

a first component being selected from natural ground calcium carbonate and/or precipitated calcium carbonate, and

a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source, and

the wet cosmetic composition is a make-up product, preferably a lip make-up product, an eye make-up product, or a facial make-up product; a nail care product, a lip care product, a skin care product, a hair care product, a hair styling product, a hair colouring product, a deodorant, a hair remover, a make-up remover, a baby care product, a sun protection product, a tanner product, a feminine hygiene product, a bath product, a facial cleaning product, a hair cleaning product, a skin cleaning product, a soap product, or an oral care product.

Advantageous embodiments of the present invention are defined in the corresponding subclaims.

According to one embodiment the first component is present in an amount from 1 wt.-% to 99 wt.-%, preferably from 30 wt.-% to 99 wt.-%, more preferably from 50 wt.-% to 95 wt.-%, even more preferably from 60 wt.-% to 95 wt.-%, and most preferably from 70 wt.-% to 90 wt.-%, based on the total weight of the mineral blend, and wherein the second component is present in an amount from 1 wt.-% to 99 wt.-%, preferably from 1 wt.-% to 70 wt.-%, more preferably from 5 wt.-% to 50 wt.-%, even more preferably from 5 wt.-% to 40 wt.-%, and most preferably from 10 wt.-% to 30 wt.-%, based on the total weight of the mineral blend. According to a further embodiment the first component is a natural ground calcium carbonate selected from the group consisting of marble, chalk, limestone, and mixtures thereof, and/or wherein the first component is a precipitated calcium carbonate selected from the group consisting of precipitated calcium carbonates having an aragonitic, vateritic or calcitic crystal form, and mixtures thereof. According to a further embodiment the at least one H3O+ ion donor is selected from the group consisting of hydrochloric acid, sulphuric acid, sulphurous acid, phosphoric acid, citric acid, oxalic acid, an acidic salt, acetic acid, formic acid, and mixtures thereof, preferably the at least one H3O+ ion donor is selected from the group consisting of hydrochloric acid, sulphuric acid, sulphurous acid, phosphoric acid, oxalic acid, H2PO4−, being at least partially neutralised by a cation selected from Li+, Na+ and/or K+, HPO42−, being at least partially neutralised by a cation selected from Li+, Na+, K+, Mg2+, and/or Ca2+, and mixtures thereof, more preferably the at least one H3O+ ion donor is selected from the group consisting of hydrochloric acid, sulphuric acid, sulphurous acid, phosphoric acid, oxalic acid, or mixtures thereof, and most preferably, the at least one H3O+ ion donor is phosphoric acid.

According to one embodiment the first component has a volume median particle size d50 from 0.1 to 50 μm, preferably from 0.5 to 40 μm, more preferably from 0.5 to 20 μm, even more preferably from 0.5 to 10 μm, and most preferably from 0.8 to 8 μm, and/or a specific surface area of from 0.5 m2/g to 30 m2/g, preferably from 1 m2/g to 20 m2/g, and more preferably from 2 m2/g to 15 m2/g, measured using nitrogen and the BET method, and/or wherein the second component has a volume median particle size d50 from 0.5 to 50 μm, preferably from 1 to 40 μm, more preferably from 1.2 to 30 μm, even more preferably from 1.5 to 15 μm, and most preferably from 3 to 10 μm, and/or a specific surface area of from 15 m2/g to 200 m2/g, preferably from 20 m2/g to 180 m2/g, more preferably from 25 m2/g to 160 m2/g, and most preferably from 30 m2/g to 90 m2/g, measured using nitrogen and the BET method.

According to one embodiment the surface-reacted calcium carbonate is associated with at least one active agent selected from pharmaceutically active agents, biologically active agents, disinfecting agents, preservatives, vitamins, flavouring agents, surfactants, oils, fragrances, and mixtures thereof. According to a further embodiment the wet cosmetic composition comprises a water content of at least 15 wt.-%, preferably at least 20 wt.-%, more preferably at least 25 wt.-%, and most preferably at least 30 wt.-%, based on the total weight of the wet cosmetic composition. According to still a further embodiment the wet cosmetic composition is free of talc or a talc-containing material. According to still a further embodiment the wet cosmetic preparation further comprises at least one additive selected from the group consisting of bleaching agents, thickeners, stabilizers, chelating agents, preserving agents, wetting agents, emulsifiers, emollients, fragrances, colorants, flavours, oils, skin tanning compounds, antioxidants, minerals, pigments, UV-A and/or UV-B filter, and mixtures thereof.

According to one embodiment the wet cosmetic preparation is in form of a paste, an ointment, a cream, a gel, a lotion, a solution, an emulsion, or a solid form. According to a further embodiment the wet cosmetic composition is a make-up product, preferably a lip make-up product, an eye make-up product, or a facial make-up product; a nail care product, a lip care product, a skin care product, a hair care product, a hair styling product, a hair colouring product, a deodorant, a hair remover, a make-up remover, a baby care product, a sun protection product, a tanner product, a feminine hygiene product, a bath product, a facial cleaning product, a hair cleaning product, a skin cleaning product, a soap product, or an oral care product. According to still a further embodiment the mineral blend is used for absorbing fluids, as fragrance booster, for decreasing skin friction, for modifying the skin feel, and/or for modifying the skin appearance. According to still a further embodiment the mineral blend is used as a replacement for talc or talc-containing materials.

According to one embodiment the wet cosmetic composition comprises the mineral blend in an amount from 0.1 to 50 wt.-%, based on the total weight of the wet cosmetic composition, preferably from 0.5 to 20 wt. %, more preferably from 1 to 10 wt.-%, and most preferably from 3 to 6 wt.-%.

It should be understood that for the purposes of the present invention, the following terms have the following meanings:

For the purpose of the present invention, the term “cosmetic composition” refers to a composition that is applied onto skin, hair or tissues of the oral cavity and that does not contain harmful and/or irritating substances, and/or does not contain substances, which are not approved for use in a cosmetic composition. Substances, which are not approved for use in cosmetics may, for example, be found in “REGULATION (EC) No 1223/2009 OF THE EUROPEAN PARLIAMENT AND OF THE COUNCIL”. A “cosmetic agent” refers to a material that can be used in a cosmetic composition, i.e. it is not harmful and/or irritating, and/or is not a substances, which is not approved for use in a cosmetic composition.

A “wet” cosmetic composition in the meaning of the present invention refers to a cosmetic composition, which contains a water content of more than 15 wt.-%, preferably more than 20 wt.-%, and more preferably more than 25 wt.-%, based on the total weight of the cosmetic composition. A suitable method for determining the water content of the wet cosmetic composition will be selected by the skilled person. For example, the water content may be determined according to the Coulometric Karl Fischer measurement method, wherein the composition is heated to 220° C., and the water content released as vapour and isolated using a stream of nitrogen gas (at 100 ml/min) is determined in a Coulometric Karl Fischer unit.

“Natural ground calcium carbonate” (GCC) in the meaning of the present invention is a calcium carbonate obtained from natural sources, such as limestone, marble, or chalk, and processed through a wet and/or dry treatment such as grinding, screening and/or fractionating, for example, by a cyclone or classifier.

“Precipitated calcium carbonate” (PCC) in the meaning of the present invention is a synthesised material, obtained by precipitation following reaction of carbon dioxide and lime in an aqueous, semi-dry or humid environment or by precipitation of a calcium and carbonate ion source in water. PCC may be in the vateritic, calcitic or aragonitic crystal form. PCCs are described, for example, in EP 2 447 213 A1, EP 2 524 898 A1, EP 2 371 766 A1, EP 1 712 597 A1, EP 1 712 523 A1, or WO 2013/142473 A1.

The term “surface-reacted” in the meaning of the present application shall be used to indicate that a material has been subjected to a process comprising partial dissolution of said material upon treatment with an H3O+ ion donor (e.g., by use of water-soluble free acids and/or acidic salts) in aqueous environment followed by a crystallization process which may occur in the absence or presence of further crystallization additives.

An “H3O+ ion donor” in the context of the present invention is a Brønsted acid and/or an acid salt, i.e. a salt containing an acidic hydrogen. The term “acid” as used herein refers to an acid in the meaning of the definition by Brønsted and Lowry (e.g., H2SO4, HSO4−). The term “free acid” refers only to those acids being in the fully protonated form (e.g., H2SO4).

The “particle size” of particulate materials is described by its distribution of particle sizes dx. Unless indicated otherwise, the value dx represents the diameter relative to which x % by weight of the particles have diameters less than dx. This means that, for example, the d20 value is the particle size at which 20 wt.-% of all particles are smaller than that particle size. The d50 value is thus the weight median particle size, i.e. 50 wt.-% of all particles are smaller than this particle size. For the purpose of the present invention, the particle size is specified as weight median particle size d50 (wt.) unless indicated otherwise. Particle sizes were determined by using a Sedigraph™ 5100 instrument or Sedigraph™ 5120 instrument of Micromeritics Instrument Corporation. The method and the instrument are known to the skilled person and are commonly used to determine the particle size of fillers and pigments. The measurements were carried out in an aqueous solution of 0.1 wt.-% Na4P2O7.

For certain materials specified herein, the “particle size” is described as volume-based particle size distribution. This is indicated, for example, by “volume based median particle size”, “volume median particle size” or “volume top cut particle size”. Volume median particle size d50 was evaluated using a Malvern Mastersizer 2000 or 3000 Laser Diffraction System. The d50 or d98 value, measured using a Malvern Mastersizer 2000 or 3000 Laser Diffraction System, preferably a Malvern Mastersizer 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005. The measurements were carried out in an aqueous solution of 0.1 wt.-% Na4P2O7.

The term “particulate” in the meaning of the present application refers to materials composed of a plurality of particles. Said plurality of particles may be defined, for example, by its particle size distribution. The expression “particulate material” may comprise granules, powders, grains, tablets, or crumbles.

The “specific surface area” (expressed in m2/g) of a material as used throughout the present document can be determined by the Brunauer Emmett Teller (BET) method with nitrogen as adsorbing gas and by use of a ASAP 2460 instrument from Micromeritics. The method is well known to the skilled person and defined in ISO 9277:2010. Prior to such measurements, the sample was filtered within a Buchner funnel, rinsed with deionised water and dried at 110° C. in an oven for at least 12 hours. The total surface area (in m2) of said material can be obtained by multiplication of the specific surface area (in m2/g) and the mass (in g) of the material.

In the context of the present invention, the term “pore” is to be understood as describing the space that is found between and/or within particles, i.e. that is formed by the particles as they pack together under nearest neighbour contact (interparticle pores), such as in a powder or a compact and/or the void space within porous particles (intraparticle pores), and that allows the passage of liquids under pressure when saturated by the liquid and/or supports absorption of surface wetting liquids.

Unless specified otherwise, the term “drying” refers to a process according to which at least a portion of water is removed from a material to be dried such that a constant weight of the obtained “dried” material at 200° C. is reached. Moreover, a “dried” or “dry” material may be defined by its total moisture content which, unless specified otherwise, is less than or equal to 1.0 wt.-%, preferably less than or equal to 0.5 wt.-%, more preferably less than or equal to 0.2 wt.-%, and most preferably between 0.03 and 0.07 wt.-%, based on the total weight of the dried material.

For the purpose of the present application, “water-insoluble” materials are defined as those which, when mixed with 100 ml of deionised water and filtered at 20° C. to recover the liquid filtrate, provide less than or equal to 0.1 g of recovered solid material following evaporation at 95 to 100° C. of 100 g of said liquid filtrate. “Water-soluble” materials are defined as materials leading to the recovery of greater than 0.1 g of solid material following evaporation at 95 to 100° C. of 100 g of said liquid filtrate. In order to assess whether a material is an insoluble or soluble material in the meaning of the present invention, the sample size is greater than 0.1 g, preferably 0.5 g or more.

A “suspension” or “slurry” in the meaning of the present invention comprises undissolved solids and water, and optionally further additives, and usually contains large amounts of solids and, thus, is more viscous and can be of higher density than the liquid from which it is formed.

The expression “skin feel” in the meaning of the present invention refers to the feeling of the skin during and/or after the application of the wet cosmetic composition onto the skin surface. For example, the skin feel may relate to a soft, smooth, greasy, dry, tight and/or flexible feeling of the skin.

The expression “skin appearance” in the meaning of the present invention relates to the optical impression of the skin to the eye of the beholder during and/or after application of the wet cosmetic composition. For example, the skin may appear shiny, matt, evenly or unevenly toned.

Where an indefinite or definite article is used when referring to a singular noun, e.g., “a”, “an” or “the”, this includes a plural of that noun unless anything else is specifically stated.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Terms like “obtainable” or “definable” and “obtained” or “defined” are used interchangeably. This, for example, means that, unless the context clearly dictates otherwise, the term “obtained” does not mean to indicate that, for example, an embodiment must be obtained by, for example, the sequence of steps following the term “obtained” though such a limited understanding is always included by the terms “obtained” or “defined” as a preferred embodiment.

Whenever the terms “including” or “having” are used, these terms are meant to be equivalent to “comprising” as defined hereinabove.

According to the present invention, the use of a mineral blend as cosmetic agent for a wet cosmetic composition is provided. The mineral blend comprises a first component being selected from natural ground calcium carbonate and/or precipitated calcium carbonate, and a second component being a surface-reacted calcium carbonate. The surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source.

In the following details and preferred embodiments of the inventive use will be set out in more details. It is to be understood that these technical details and embodiments also apply to the inventive composition.

The First Component

According to the present invention, the mineral blend used as cosmetic agent for a wet cosmetic composition comprises a first component being a natural ground calcium carbonate and/or a precipitated calcium carbonate.

According to one embodiment, the first component has a volume median particle size d50 from 0.1 to 50 μm, preferably from 0.5 to 40 μm, more preferably from 0.5 to 20 μm, even more preferably from 0.5 to 10 μm, and most preferably from 0.8 to 8 μm. In addition or alternatively, the first component may have a specific surface area of from 0.5 m2/g to 30 m2/g, preferably from 1 m2/g to 20 m2/g, and more preferably from 2 m2/g to 15 m2/g, measured using nitrogen and the BET method.

According to another embodiment, the first component has a volume median particle size d50 from 0.1 to 50 μm, preferably from 0.5 to 40 μm, more preferably from 0.5 to 20 μm, even more preferably from 0.5 to 10 μm, and most preferably from 0.8 to 8 μm, and the first component has a specific surface area of from 0.5 m2/g to 30 m2/g, preferably from 1 m2/g to 20 m2/g, and more preferably from 2 m2/g to 15 m2/g, measured using nitrogen and the BET method.

According to one preferred embodiment, the first component is a natural ground calcium carbonate. It is appreciated that the natural ground calcium carbonate can be one specific natural ground calcium carbonate or a mixture of different kinds of natural ground calcium carbonate(s).

According to one embodiment of the present invention, the natural ground calcium carbonate comprises, preferably consists of, one kind of natural ground calcium carbonate. Alternatively, the natural ground calcium carbonate comprises, preferably consists of, two or more kinds of natural ground calcium carbonates. For example, the natural ground calcium carbonate comprises, preferably consists of, two or three kinds of natural ground calcium carbonates. Preferably, the natural ground calcium carbonate comprises, more preferably consists of, one kind of natural ground calcium carbonate.

According to one embodiment of the present invention, the natural ground calcium carbonate is selected from the group consisting of marble, chalk, limestone, and mixtures thereof. More preferably, the natural ground calcium carbonate may be selected from limestone and/or marble, and most preferably the natural ground calcium carbonate is marble.

A natural ground calcium carbonate may be obtained, for example, in a wet and/or dry comminution step, such as crushing and/or grinding, from natural calcium carbonate-containing minerals (e.g. chalk, limestone, marble or dolomite). According to one embodiment, the natural ground calcium carbonate is a wet-natural ground calcium carbonate. In another embodiment, the natural ground calcium carbonate is a dry-natural ground calcium carbonate.

The grinding step can be carried out with any conventional grinding device, for example, under conditions such that refinement predominantly results from impacts with a secondary body, i.e. in one or more of a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. The grinding step may also be performed under conditions such that autogenous grinding takes place and/or by horizontal ball milling, and/or other such processes known to the skilled man.

In one embodiment, grinding is carried out in a vertical or horizontal ball mill, preferably in a vertical ball mill. Such vertical and horizontal ball mills usually consist of a vertically or horizontally arranged, cylindrical grinding chamber comprising an axially fast rotating agitator shaft being equipped with a plurality of paddles and/or stirring discs, such as described for example in EP0607840 A1.

It is to be noted that grinding of the calcium carbonate-containing mineral may be carried out by using at least one of the aforementioned grinding methods or devices. However, it is also possible to use a combination of any of the foregoing methods or a series of any of the aforementioned grinding devices.

Subsequent to the grinding step, the ground calcium carbonate-containing mineral may, optionally, be divided into two or more fractions, each having different particle distributions, by use of a classifying step. A classifying step in general serves to divide a feed fraction having a certain particle size distribution into a coarse fraction, which may be subjected to another grinding cycle, and a fine fraction, which may be used as the final product. For this purpose, screening devices as well as gravity-based devices, such as centrifuges or cyclones (e.g. hydrocyclones) and any combination of the aforementioned devices may be used.

In case the first component of the mineral blend according to the present invention is a natural ground calcium carbonate, the natural ground calcium carbonate may have specific physical characteristics such as a specific particle size and/or a specific surface area.

According to one embodiment, the natural ground calcium carbonate has a volume median particle size d50 from 0.1 to 50 μm, preferably from 0.5 to 40 μm, more preferably from 0.5 to 20 μm, even more preferably from 0.5 to 10 μm, and most preferably from 0.8 to 8 μm, and/or a volume top cut particle size d98 of from 2 to 80 μm, preferably from 2 to 60 μm, more preferably 2 to 40 μm, even more preferably from 3 to 30 μm, and most preferably from 4 to 20 μm.

According to one embodiment, the natural ground calcium carbonate has a specific surface area of from 0.5 m2/g to 30 m2/g, preferably from 1 m2/g to 20 m2/g, and more preferably from 2 m2/g to 15 m2/g, measured using nitrogen and the BET method.

According to one preferred embodiment, the first component is a natural ground calcium carbonate having a volume median particle size d50 from 0.1 to 50 μm, preferably from 0.5 to 40 μm, more preferably from 0.5 to 20 μm, even more preferably from 0.5 to 10 μm, and most preferably from 0.8 to 8 μm, and a volume top cut particle size d98 of from 2 to 80 μm, preferably from 2 to 60 μm, more preferably 2 to 40 μm, even more preferably from 3 to 30 μm, and most preferably from 4 to 20 μm, and a specific surface area of from 0.5 m2/g to 30 m2/g, preferably from 1 m2/g to 20 m2/g, and more preferably from 2 m2/g to 15 m2/g, measured using nitrogen and the BET method.

In one embodiment, the natural ground calcium carbonate has a volume median particle size d₅₀ from 0.8 to 8 μm, preferably from 1 to 5 μm, and a volume top cut particle size d₉₈ of from 4 to 20 μm, and a specific surface area of from 1 m²/g to 20 m²/g, 2 m²/g to 15 m²/g, measured using nitrogen and the BET method.

According to another embodiment, the first component is a precipitated calcium carbonate.

It is appreciated that the precipitated calcium carbonate can be one or a mixture of different kinds of precipitated calcium carbonate(s).

According to one embodiment of the present invention, the precipitated calcium carbonate comprises, preferably consists of, one kind of precipitated calcium carbonate. Alternatively, the precipitated calcium carbonate comprises, preferably consists of, two or more kinds of precipitated calcium carbonate(s). For example, the precipitated calcium carbonate comprises, preferably consists of, two or three kinds of precipitated calcium carbonates. Preferably, the precipitated calcium carbonate comprises, more preferably consists of, one kind of precipitated calcium carbonate.

According to one embodiment, the precipitated calcium carbonate is selected from the group consisting of precipitated calcium carbonates having an aragonitic, vateritic or calcitic crystal form, and mixtures thereof.

It is appreciated that the precipitated calcium carbonate may have specific physical characteristics such as a specific particle size or a specific surface area.

According to one embodiment, the precipitated calcium carbonate has a volume median particle size d50 from 0.1 to 50 μm, preferably from 0.5 to 40 μm, more preferably from 0.5 to 20 μm, even more preferably from 0.5 to 10 μm, and most preferably from 0.8 to 8 μm. According to another embodiment, the precipitated calcium carbonate has a volume median particle size d50 from 0.25 to 50 μm, more preferably from 0.3 to 10 μm, and most preferably from 0.4 to 7 μm. In one embodiment, the precipitated calcium carbonate has a volume top cut particle size d98 of from 1 to 100 μm, preferably of from 1 to 50 μm, more preferably of from 1.5 to 30 μm, and most preferably of from 1.5 to 20 μm. According to another embodiment, the precipitated calcium carbonate has a volume median particle size d50 from 0.3 to 10 μm, and most preferably from 0.4 to 7 μm and a volume top cut particle size d98 of from 1.5 to 30 μm, and most preferably of from 1.5 to 20 μm.

According to another embodiment, the precipitated calcium carbonate has a specific surface area of from 0.5 m2/g to 30 m2/g, preferably from 1 m2/g to 20 m2/g, and most preferably from 2 m2/g to 15 m2/g, measured using nitrogen and the BET method.

According to another embodiment, the precipitated calcium carbonate has a volume median particle size d50 from 0.3 to 10 μm, and most preferably from 0.4 to 7 μm and a volume top cut particle size d98 of from 1.5 to 30 μm, and most preferably of from 1.5 to 20 μm, and a specific surface area of from 0.5 m2/g to 30 m2/g, preferably from 1 m2/g to 20 m2/g, and most preferably from 2 m2/g to 15 m2/g measured using nitrogen and the BET method.

According to one embodiment, the first component is selected from natural ground calcium carbonate and precipitated calcium carbonate. In other words, the first component is a mixture of at least one natural ground calcium carbonate and at least one precipitated calcium carbonate.

The Second Component

According to the present invention, the mineral blend used as cosmetic agent for a wet cosmetic composition comprises a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source.

It is appreciated that the surface-reacted calcium carbonate can be one or a mixture of different kinds of surface-reacted calcium carbonate(s).

In one embodiment of the present invention, the surface-reacted calcium carbonate comprises, preferably consists of, one kind of surface-reacted calcium carbonate. Alternatively, the surface-reacted calcium carbonate comprises, preferably consists of, two or more kinds of surface-reacted calcium carbonates. For example, the surface-reacted calcium carbonate comprises, preferably consists of, two or three kinds of surface-reacted calcium carbonates. Preferably, the surface-reacted calcium carbonate comprises, more preferably consists of, one kind of surface-reacted calcium carbonate.

In a preferred embodiment of the invention the surface-reacted calcium carbonate is obtained by a process comprising the steps of: (a) providing a suspension of natural or precipitated calcium carbonate, (b) adding at least one acid having a pKa value of 0 or less at 20° C. or having a pKa value from 0 to 2.5 at 20° C. to the suspension of step (a), and (c) treating the suspension of step (a) with carbon dioxide before, during or after step (b). According to another embodiment the surface-reacted calcium carbonate is obtained by a process comprising the steps of: (A) providing a natural or precipitated calcium carbonate, (B) providing at least one water-soluble acid, (C) providing gaseous CO2, (D) contacting said natural or precipitated calcium carbonate of step (A) with the at least one acid of step (B) and with the CO₂ of step (C), characterised in that: (i) the at least one acid of step B) has a pKa of greater than 2.5 and less than or equal to 7 at 20° C., associated with the ionisation of its first available hydrogen, and a corresponding anion is formed on loss of this first available hydrogen capable of forming a water-soluble calcium salt, and (ii) following contacting the at least one acid with natural or precipitated calcium carbonate, at least one water-soluble salt, which in the case of a hydrogen-containing salt has a pKa of greater than 7 at 20° C., associated with the ionisation of the first available hydrogen, and the salt anion of which is capable of forming water-insoluble calcium salts, is additionally provided.

“Natural ground calcium carbonate” (GCC) preferably is selected from calcium carbonate containing minerals selected from the group comprising marble, chalk, limestone and mixtures thereof. Natural calcium carbonate may comprise further naturally occurring components such as alumino silicate etc.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

“Precipitated calcium carbonate” (PCC) in the meaning of the present invention is a synthesized material, generally obtained by precipitation following reaction of carbon dioxide and calcium hydroxide in an aqueous environment or by precipitation of calcium and carbonate ions, for example CaCl2 and Na2CO3, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

Precipitated calcium carbonate may be ground prior to the treatment with carbon dioxide and at least one H3O+ ion donor by the same means as used for grinding natural calcium carbonate as described above.

According to one embodiment of the present invention, the natural or precipitated calcium carbonate is in form of particles having a weight median particle size d50 of 0.05 to 10.0 μm, preferably 0.2 to 5.0 μm, more preferably 0.4 to 3.0 μm. According to a further embodiment of the present invention, the natural or precipitated calcium carbonate is in form of particles having a weight top cut particle size d98 of 0.15 to 30 μm, preferably 0.6 to 15 μm, more preferably 1.2 to 10 μm, most preferably 1.5 to 4 μm, especially 1.6 μm.

The natural and/or precipitated calcium carbonate may be used dry or suspended in water. Preferably, a corresponding slurry has a content of natural or precipitated calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H3O+ ion donor used for the preparation of surface reacted calcium carbonate may be any strong acid, medium-strong acid, or weak acid, or mixtures thereof, generating H3O+ ions under the preparation conditions. According to the present invention, the at least one H3O+ ion donor can also be an acidic salt, generating H3O+ ions under the preparation conditions.

According to one embodiment, the at least one H3O+ ion donor is a strong acid having a pKa of 0 or less at 20° C.

According to another embodiment, the at least one H3O+ ion donor is a medium-strong acid having a pKa value from 0 to 2.5 at 20° C. If the pKa at 20° C. is 0 or less, the acid is preferably selected from sulphuric acid, hydrochloric acid, or mixtures thereof. If the pKa at 20° C. is from 0 to 2.5, the H3O+ ion donor is preferably selected from H2S03, H3PO4, oxalic acid, or mixtures thereof. The at least one H3O+ ion donor can also be an acidic salt, for example, HSO4− or H2PO4−, being at least partially neutralized by a corresponding cation such as Li+, Na+ or K+, or HPO42−, being at least partially neutralised by a corresponding cation such as Li+, Na+, K+, Mg2+ or Ca2+. The at least one H3O+ ion donor can also be a mixture of one or more acids and one or more acidic salts.

According to still another embodiment, the at least one H3O+ ion donor is a weak acid having a pKa value of greater than 2.5 and less than or equal to 7, when measured at 20° C., associated with the ionisation of the first available hydrogen, and having a corresponding anion, which is capable of forming water-soluble calcium salts. Subsequently, at least one water-soluble salt, which in the case of a hydrogen-containing salt has a pKa of greater than 7, when measured at 20° C., associated with the ionisation of the first available hydrogen, and the salt anion of which is capable of forming water-insoluble calcium salts, is additionally provided. According to the preferred embodiment, the weak acid has a pKa value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the at least one H3O+ ion donor is selected from the group consisting of hydrochloric acid, sulphuric acid, sulphurous acid, phosphoric acid, citric acid, oxalic acid, acetic acid, formic acid, and mixtures thereof. Preferably the at least one H3O+ ion donor is selected from the group consisting of hydrochloric acid, sulphuric acid, sulphurous acid, phosphoric acid, oxalic acid, H2PO4−, being at least partially neutralised by a corresponding cation such as Li+, Na+ or K+, HPO42−, being at least partially neutralised by a corresponding cation such as Li+, Na+, K+, Mg2+, or Ca2+ and mixtures thereof, more preferably the at least one acid is selected from the group consisting of hydrochloric acid, sulphuric acid, sulphurous acid, phosphoric acid, oxalic acid, or mixtures thereof, and most preferably, the at least one H3O+ ion donor is phosphoric acid.

The one or more H3O+ ion donor can be added to the suspension as a concentrated solution or a more diluted solution. Preferably, the molar ratio of the H3O+ ion donor to the natural or precipitated calcium carbonate is from 0.01 to 4, more preferably from 0.02 to 2, even more preferably 0.05 to 1 and most preferably 0.1 to 0.58.

As an alternative, it is also possible to add the H3O+ ion donor to the water before the natural or precipitated calcium carbonate is suspended.

In a next step, the natural or precipitated calcium carbonate is treated with carbon dioxide. If a strong acid such as sulphuric acid or hydrochloric acid is used for the H3O+ ion donor treatment of the natural or precipitated calcium carbonate, the carbon dioxide is automatically formed. Alternatively or additionally, the carbon dioxide can be supplied from an external source.

H3O+ ion donor treatment and treatment with carbon dioxide can be carried out simultaneously which is the case when a strong or medium-strong acid is used. It is also possible to carry out H3O+ ion donor treatment first, e.g. with a medium strong acid having a pKa in the range of 0 to 2.5 at 20° C., wherein carbon dioxide is formed in situ, and thus, the carbon dioxide treatment will automatically be carried out simultaneously with the H3O+ ion donor treatment, followed by the additional treatment with carbon dioxide supplied from an external source.

In a preferred embodiment, the H3O+ ion donor treatment step and/or the carbon dioxide treatment step are repeated at least once, more preferably several times. According to one embodiment, the at least one H3O+ ion donor is added over a time period of at least about 5 min, preferably at least about 10 min, typically from about 10 to about 20 min, more preferably about 30 min, even more preferably about 45 min, and sometimes about 1 h or more.

Subsequent to the H3O+ ion donor treatment and carbon dioxide treatment, the pH of the aqueous suspension, measured at 20° C., naturally reaches a value of greater than 6.0, preferably greater than 6.5, more preferably greater than 7.0, even more preferably greater than 7.5, thereby preparing the surface-reacted natural or precipitated calcium carbonate as an aqueous suspension having a pH of greater than 6.0, preferably greater than 6.5, more preferably greater than 7.0, even more preferably greater than 7.5.

In a particular preferred embodiment the surface reacted calcium carbonate is a reaction product of natural ground calcium carbonate (GNCC) with carbon dioxide and phosphoric acid, wherein the carbon dioxide is formed in situ by the phosphoric acid treatment.

Further details about the preparation of the surface-reacted natural calcium carbonate are disclosed in WO0039222 A1, WO2004083316 A1, WO2005121257 A2, WO2009074492 A1, EP2264108 A1, EP2264109 A1 and US20040020410 A1, the content of these references herewith being included in the present application.

Similarly, surface-reacted precipitated calcium carbonate is obtained. As can be taken in detail from WO2009074492 A1, surface-reacted precipitated calcium carbonate is obtained by contacting precipitated calcium carbonate with H3O+ ions and with anions being solubilized in an aqueous medium and being capable of forming water-insoluble calcium salts, in an aqueous medium to form a slurry of surface-reacted precipitated calcium carbonate, wherein said surface-reacted precipitated calcium carbonate comprises an insoluble, at least partially crystalline calcium salt of said anion formed on the surface of at least part of the precipitated calcium carbonate.

Said solubilized calcium ions correspond to an excess of solubilized calcium ions relative to the solubilized calcium ions naturally generated on dissolution of precipitated calcium carbonate by H3O+ ions, where said H3O+ ions are provided solely in the form of a counterion to the anion, i.e. via the addition of the anion in the form of an acid or non-calcium acid salt, and in absence of any further calcium ion or calcium ion generating source.

Said excess solubilized calcium ions are preferably provided by the addition of a soluble neutral or acid calcium salt, or by the addition of an acid or a neutral or acid non-calcium salt which generates a soluble neutral or acid calcium salt in situ.

Said H3O+ ions may be provided by the addition of an acid or an acid salt of said anion, or the addition of an acid or an acid salt which simultaneously serves to provide all or part of said excess solubilized calcium ions.

In a further preferred embodiment of the preparation of the surface-reacted natural or precipitated calcium carbonate, the natural or precipitated calcium carbonate is reacted with the one or more H3O+ ion donors and/or the carbon dioxide in the presence of at least one compound selected from the group consisting of silicate, silica, aluminium hydroxide, earth alkali aluminate such as sodium or potassium aluminate, magnesium oxide, or mixtures thereof. Preferably, the at least one silicate is selected from an aluminium silicate, a calcium silicate, or an earth alkali metal silicate. These components can be added to an aqueous suspension comprising the natural or precipitated calcium carbonate before adding the one or more H3O+ ion donors and/or carbon dioxide.

Alternatively, the silicate and/or silica and/or aluminium hydroxide and/or earth alkali aluminate and/or magnesium oxide component(s) can be added to the aqueous suspension of natural or precipitated calcium carbonate while the reaction of natural or precipitated calcium carbonate with the one or more H3O+ ion donors and carbon dioxide has already started. Further details about the preparation of the surface-reacted natural or precipitated calcium carbonate in the presence of at least one silicate and/or silica and/or aluminium hydroxide and/or earth alkali aluminate component(s) are disclosed in WO2004083316 A1, the content of this reference herewith being included in the present application.

The surface-reacted calcium carbonate can be kept in suspension, optionally further stabilised by a dispersant. Conventional dispersants known to the skilled person can be used. A preferred dispersant is comprised of polyacrylic acids and/or carboxymethylcelluloses.

Alternatively, the aqueous suspension described above can be dried, thereby obtaining the solid (i.e. dry or containing as little water that it is not in a fluid form) surface-reacted natural or precipitated calcium carbonate in the form of granules or a powder.

According to one embodiment, the surface-reacted calcium carbonate has a specific surface area of from 15 m2/g to 200 m2/g, preferably from 20 m2/g to 180 m2/g, and more preferably from 25 m2/g to 160 m2/g, and most preferably from 30 m2/g to 90 m2/g measured using nitrogen and the BET method. The BET specific surface area in the meaning of the present invention is defined as the surface area of the particles divided by the mass of the particles. As used therein the specific surface area is measured by adsorption using the BET isotherm (ISO 9277:2010) and is specified in m2/g.

According to one embodiment the surface-reacted calcium carbonate has a volume median particle size d50 from 0.5 to 50 μm, preferably from 1 to 40 μm, more preferably from 1.2 to 30 μm, even more preferably from 1.5 to 15 μm, and most preferably from 3 to 10 μm.

It may furthermore be preferred that the surface-reacted calcium carbonate particles have a volume top cut particle size d98 of from 2 to 150 μm, preferably from 4 to 100 μm, more preferably 6 to 80 μm, even more preferably from 8 to 60 μm, even more preferably from 8 to 30 μm, and most preferably from 12 to 25 μm.

The value dx represents the diameter relative to which x % of the particles have diameters less than dx. This means that the d98 value is the particle size at which 98% of all particles are smaller. The d98 value is also designated as “top cut”. The dx values may be given in volume or weight percent. The d50 (wt) value is thus the weight median particle size, i.e. 50 wt.-% of all grains are smaller than this particle size, and the d50 (vol) value is the volume median particle size, i.e. 50 vol. % of all grains are smaller than this particle size.

Volume median grain diameter d50 was evaluated using a Malvern Mastersizer 2000 Laser Diffraction System or a Malvern Mastersizer 3000 Laser Diffraction System. The d50 or d98 value, measured using a Malvern Mastersizer 2000 Laser Diffraction System or a Malvern Mastersizer 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005. The measurements were carried out in an aqueous solution of 0.1 wt.-% Na4P2O7.

The weight median grain diameter is determined by the sedimentation method, which is an analysis of sedimentation behaviour in a gravimetric field. The measurement is made with a Sedigraph™ 5100 or 5120, Micromeritics Instrument Corporation. The method and the instrument are known to the skilled person and are commonly used to determine grain size of fillers and pigments. The measurement is carried out in an aqueous solution of 0.1 wt.-% Na4P2O7. The samples were dispersed using a high speed stirrer and sonicated.

The processes and instruments are known to the skilled person and are commonly used to determine grain size of fillers and pigments.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (— nm). The equilibration time used at each pressure step is 20 seconds. The sample material is sealed in a 5 cm3 chamber powder penetrometer for analysis. The data are corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p. 1753-1764).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine interparticle packing of the particles themselves. If they also have intraparticle pores, then this region appears bi-modal, and by taking the specific pore volume intruded by mercury into pores finer than the modal turning point, i.e. finer than the bi-modal point of inflection, the specific intraparticle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

Preferably, the surface-reacted calcium carbonate has an intra-particle intruded specific pore volume in the range from 0.1 to 2.3 cm3/g, more preferably from 0.2 to 2.0 cm3/g, especially preferably from 0.4 to 1.8 cm3/g and most preferably from 0.6 to 1.6 cm3/g, calculated from mercury porosimetry measurement.

The intra-particle pore size of the surface-reacted calcium carbonate preferably is in a range of from 0.004 to 1.6 μm, more preferably in a range of between 0.005 to 1.3 μm, especially preferably from 0.006 to 1.15 μm and most preferably of 0.007 to 1.0 μm, e.g. 0.004 to 0.16 μm determined by mercury porosimetry measurement.

According to an exemplary embodiment, the surface-reacted calcium carbonate has a volume median particle size d50 from 1.5 to 15 μm, preferably from 4 to 8 μm; a specific surface-area of from 30 to 140 m2/g, preferably from 40 to 100 m2/g, more preferably from 50 to 80 m2/g, measured using nitrogen and the BET method; and an intra-particle intruded specific pore volume from 0.2 to 2.0 cm3/g, preferably from 0.6 to 1.6 cm3/g, calculated from mercury porosimetry measurement. According to another exemplary embodiment, the surface-reacted calcium carbonate has a volume median particle size d50 from 5 to 9 μm; a specific surface-area of from 45 to 80 m2/g, measured using nitrogen and the BET method; and a volume top cut particle size d98 of from 10 to 20 μm.

Due to the intra and interpore structure of the surface-reacted calcium carbonate, it can be a superior agent to deliver previously adsorbed and/or absorbed materials over time relative to common materials having similar specific surface areas. Thus, generally, any agent fitting into the intra- and/or inter particle pores of the surface-reacted calcium carbonate is suitable to be transported by the surface-reacted calcium carbonate according to the invention. For example, active agents such as those selected from the group comprising pharmaceutically active agents, biologically active agents, disinfecting agents, preservatives, vitamins, flavouring agents, surfactants, oils, fragrances, and mixtures thereof can be used. According to one embodiment, at least one active agent is associated with the surface-reacted calcium carbonate.

According to one embodiment of the present invention, the surface-reacted calcium carbonate comprises an water-insoluble, at least partially crystalline calcium salt of an anion of the at least one acid, which is formed on the surface of the natural ground calcium carbonate or precipitated calcium carbonate. According to one embodiment, the water-insoluble, at least partially crystalline salt of an anion of the at least one acid covers the surface of the natural ground calcium carbonate or precipitated calcium carbonate at least partially, preferably completely. Depending on the employed at least one acid, the anion may be sulphate, sulphite, phosphate, citrate, oxalate, acetate, formiate and/or chloride.

According to one embodiment the surface-reacted calcium carbonate comprises:

(i) a specific surface area of from 15 to 200 m²/g measured using nitrogen and the BET method according to ISO 9277:2010, and

(ii) an intra-particle intruded specific pore volume from 0.1 to 2.3 cm³/g calculated from mercury porosimetry measurement.

The Wet Cosmetic Composition

The present invention is directed to the use of a mineral blend as cosmetic agent for a wet cosmetic composition, wherein the mineral blend comprises a first component being selected from natural ground calcium carbonate and/or precipitated calcium carbonate, and a second component being a surface-reacted calcium carbonate as defined herein.

The wet cosmetic composition may be present in any suitable form having a water content of at least 15 wt.-%, based on the total weight of the wet cosmetic composition. For example, the wet cosmetic composition may be in form of a paste, an ointment, a cream, a gel, a lotion, a solution, an emulsion, or a solid form. According to one embodiment, the wet cosmetic composition is a make-up product, a nail care product, a lip care product, a skin care product, a hair care product, a hair styling product, a hair colouring product, a deodorant, a hair remover, a make-up remover, a baby care product, a sun protection product, a tanner product, a feminine hygiene product, a bath product, a facial cleaning product, a hair cleaning product, a skin cleaning product, a soap product, or an oral care product.

According to a preferred embodiment, the make-up product is a lip make-up product, an eye make-up product, or a facial make-up product. Examples of a lip make-up product are a lipstick, a lip liner, or a lip gloss. Examples of a nail care product are a nail cream, a nail strengthener, or an anti-chew treatener. Examples of a lip care product are a lip balm, a lip serum, a lip mask, a lip scrub, a lip moisturizer, a lip oil, or a lip butter. Examples of a skin care product are a skin mask, a skin cream, a skin moisturizer, a skin oil, a skin serum, an anti-wrinkle cream, a skin gel, a body lotion, or a skin tonic.

Examples of a hair care product are a hair conditioner, a leave-in hair conditioner, or a hair mask. Examples of a hair styling product are a voluminizing spray, an anti-frizz serum, a hair mousse, a hair gel, or a hair spray. Examples of a hair colouring product are a hair colour, a hair colour cream, a hair colour spray, a hair concealer spray, a hair make-up, or a hair colour remover.

According to a preferred embodiment, the wet cosmetic composition is a deodorant. Examples of a deodorant are an antiperspirant spray, an antiperspirant cream, an antiperspirant gel, an antiperspirant gel for a roll-on container, a deodorant spray, a deodorant cream, a deodorant gel, or a deodorant liquid for a roll-on container.

Examples of a hair remover are a hair removal lotion, a hair removal cream, or a hair removal gel. Examples of a make-up remover are make-up remover water, make-up remover gel, make-up remover lotion, make-up remover oil, oil-free make up remover, or make-up remover balm.

Examples of a baby care product are baby cream, diaper rash cream, or baby wash cream.

Examples of a sun protection product are a sun protection cream, a sun protection lotion, a sun blocker, a sun protection lip balm, or a sun protection spray. Examples of a tanner product are a suntan lotion, a suntan cream, or a suntan spray. Examples of a feminine hygiene product are a vaginal cleanser or a vaginal cream. Examples of a bath product are a bubble bath or a bath oil.

Examples of a facial cleaning product are a face wash, a face cleansing oil, a face jelly cleanser, a face cleanser foam, a face cleansing gel, a face peeling, or a face scrub. Examples of a hair cleaning product are a shampoo, a hair cleaning cream, or a hair cleaning gel. Examples of a skin cleaning product are skin wash, shower lotion, a skin cleansing oil, a skin jelly cleanser, a skin cleanser foam, a face cleansing gel, a face peeling, or a face scrub. Examples of a soap product are liquid soap, gelly soap, or soap bar. Examples of an oral care product is a toothpaste, a mouthwash, or a chewable gum.

According to a further aspect of the present invention, a wet cosmetic composition comprising a mineral blend is provided, wherein the mineral blend comprises

a first component being selected from natural ground calcium carbonate and/or precipitated calcium carbonate, and

a second component being a surface-reacted calcium carbonate, wherein the surface-reacted calcium carbonate is a reaction product of natural ground calcium carbonate or precipitated calcium carbonate with carbon dioxide and at least one H₃O⁺ ion donor, wherein the carbon dioxide is formed in situ by the at least one H₃O⁺ ion donor treatment and/or is supplied from an external source, and

the wet cosmetic composition is a make-up product, preferably a lip make-up product, an eye make-up product, or a facial make-up product; a nail care product, a lip care product, a skin care product, a hair care product, a hair styling product, a hair colouring product, a deodorant, a hair remover, a make-up remover, a baby care product, a sun protection product, a tanner product, a feminine hygiene product, a bath product, a facial cleaning product, a hair cleaning product, a skin cleaning product, a soap product, or an oral care product. According to a preferred embodiment, the wet cosmetic composition is a skin care product, a deodorant, or a baby care product.

The mineral blend may comprise the first component and the second component in specific weight amounts with respect to each other. According to one embodiment, the first component is present in an amount of from 1 wt.-% to 99 wt.-%, preferably from 30 wt.-% to 99 wt.-%, more preferably from 50 wt.-% to 95 wt.-%, even more preferably from 60 wt.-% to 95 wt.-%, and most preferably from 70 wt.-% to 90 wt.-%, based on the total weight of the mineral blend, and the second component is present in an amount of from 1 wt.-% to 99 wt.-%, preferably from 1 wt.-% to 70 wt.-%, more preferably from 5 wt.-% to 50 wt.-%, even more preferably from 5 wt.-% to 40 wt.-%, and most preferably from 10 wt.-% to 30 wt.-%, based on the total weight of the mineral blend. According to another preferred embodiment, the first component is present in an amount of from 85 wt.-% to 95 wt.-%, based on the total weight of the mineral blend, and the second component is present in an amount of from 5 wt.-% to 15 wt.-%, based on the total weight of mineral blend.

The mineral blend used as cosmetic agent in the wet cosmetic composition may be present in the cosmetic composition in an amount of equal to or less than 85 wt.-%, based on the total weight of the wet cosmetic composition. According to one embodiment, the wet cosmetic composition comprises the mineral blend in an amount from 1 to 85 wt.-%, preferably from 2 to 80 wt.-%, more preferably from 5 to 70 wt.-%, even more preferably from 10 to 60 wt.-%, and most preferably from 15 to 50 wt.-%, based on the total weight of the wet cosmetic composition. According to another embodiment, the wet cosmetic composition comprises the mineral blend in an amount from 0.1 to 50 wt.-%, preferably from 0.5 to 20 wt.-%, more preferably from 1 to 10 wt.-%, and most preferably from 3 to 6 wt.-%, based on the total weight of the wet cosmetic composition.

According to a preferred embodiment, the mineral blend comprises a first component being a natural ground calcium carbonate, and a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source. According to another preferred embodiment, the mineral blend comprises a first component being a natural ground calcium carbonate, and a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source, and wherein the at least one H3O+ ion donors is phosphoric acid.

According to a preferred embodiment, the mineral blend comprises a first component being a natural ground calcium carbonate, and a second component being a surface-reacted calcium carbonate, wherein the first component has a volume median particle size d50 from 0.5 to 10 μm, preferably from 0.8 to 8 μm, and a specific surface area of from 1 m2/g to 20 m2/g, preferably from 2 m2/g to 15 m2/g, measured using nitrogen and the BET method, and wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source, wherein the at least one H3O+ ion donors is phosphoric acid, and wherein the second component has a volume median particle size d50 of from 1.5 to 15 μm, preferably from 3 to 10 μm, and a specific surface area of from 25 m2/g to 160 m2/g, preferably from 30 m2/g to 90 m2/g, measured using nitrogen and the BET method.

According to another preferred embodiment, the mineral blend comprises a first component being a natural ground calcium carbonate, and a second component being a surface-reacted calcium carbonate, wherein the first component has a volume median particle size d50 from 0.5 to 10 μm, preferably from 0.8 to 8 μm, and a specific surface area of from 1 m2/g to 20 m2/g, preferably from 2 m2/g to 15 m2/g, measured using nitrogen and the BET method, and wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source, wherein the at least one H3O+ ion donors is phosphoric acid, and wherein the second component has a volume median particle size d50 of from 1.5 to 15 μm, preferably from 3 to 10 μm, and a specific surface area of from 25 m2/g to 160 m2/g, preferably from 30 m2/g to 90 m2/g, measured using nitrogen and the BET method, wherein the first component is present in an amount of from 50 wt.-% to 95 wt.-%, preferably from 60 to 95 wt.-%, based on the total weight of the mineral blend, and the second component is present in an amount of from 5 wt.-% to 50 wt.-%, preferably from 5 wt.-% to 40 wt.-%, based on the total weight of the mineral blend, and wherein the mineral blend is present in an amount of from 0.1 to 50 wt.-%, based on the total weight of the wet cosmetic composition.

According to another embodiment, the mineral blend comprises a first component being a precipitated calcium carbonate, and a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source.

According to a further embodiment, the mineral blend comprises a first component being a precipitated calcium carbonate, and a second component being a surface-reacted calcium carbonate, wherein the first component has a volume median particle size d50 from 0.3 to 10 μm, preferably from 0.4 to 7 μm, and a specific surface area of from 3 m2/g to 35 m2/g, preferably from 4 m2/g to 25 m2/g, measured using nitrogen and the BET method, and wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source, wherein the at least one H3O+ ion donors is phosphoric acid, and wherein the second component has a volume median particle size d50 of from 1.5 to 15 μm, preferably from 3 to 10 μm, and a specific surface area of from 25 m2/g to 160 m2/g, preferably from 30 m2/g to 100 m2/g, measured using nitrogen and the BET method.

According to a further embodiment, the mineral blend comprises a first component being a precipitated calcium carbonate, and a second component being a surface-reacted calcium carbonate, wherein the first component has a volume median particle size d50 from 0.3 to 10 μm, preferably from 0.4 to 7 μm, and a specific surface area of from 3 m2/g to 35 m2/g, preferably from 4 m2/g to 25 m2/g, measured using nitrogen and the BET method, and wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source, wherein the at least one H3O+ ion donors is phosphoric acid, and wherein the second component has a volume median particle size d50 of from 1.5 to 15 μm, preferably from 3 to 10 μm, and a specific surface area of from 25 m2/g to 160 m2/g, preferably from 30 m2/g to 90 m2/g, measured using nitrogen and the BET method, wherein the first component is present in an amount of from 50 wt.-% to 90 wt. %, preferably from 70 to 90 wt.-%, based on the total weight of the mineral blend, and the second component is present in an amount of from 5 wt.-% to 50 wt.-%, preferably from 5 wt.-% to 40 wt.-%, based on the total weight of the mineral blend, and wherein the mineral blend is present in an amount of from 0.1 to 50 wt.-%, based on the total weight of the wet cosmetic composition.

It was surprisingly found by the inventors of the present invention that a mineral blend comprising a first component being selected from natural ground calcium carbonate and/or precipitated calcium carbonate and a second component being a surface-reacted calcium carbonate can be used as cosmetic agent in wet cosmetic compositions. It was surprisingly found that the inventive mineral blend may provide as a replacement for talc or talc-containing materials. Talc is a common ingredient of cosmetic compositions, in which it is used for providing the cosmetic composition with specific characteristics such as humidity absorbing characteristics and a soft touch. The inventors surprisingly found that the wet cosmetic composition of the present invention shows similar, or even improved, characteristics, compared to known talc-containing wet cosmetic compositions.

In addition, it was surprisingly found that that the mineral blend can improve the sensory properties of wet cosmetic compositions. For example, it was found that a wet cosmetic composition comprising the inventive mineral blend is less greasy and sticky compared to a wet cosmetic composition having the same ingredients except for the mineral blend. Moreover, it was found that a wet cosmetic composition comprising the inventive mineral blend can spread more easily, form a more uniform film and dries faster, when applied to the skin. The inventor of the present invention also found that the mineral blend may provide a long-lasting scent and odour control in deodorants. In other words, the mineral blend may be used as fragrance booster.

According to one embodiment, the mineral blend is used as a replacement for talc or talc-containing materials. Thus, use of a mineral blend as a replacement for talc or talc-containing materials for a wet cosmetic composition is provided, wherein the mineral blend comprises a first component being selected from natural ground calcium carbonate and/or precipitated calcium carbonate, and a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source.

According to another embodiment, the mineral blend is used for absorbing fluids, as fragrance booster, for decreasing skin friction, for modifying skin feel, and/or for modifying the skin appearance. Thus, use of a mineral blend for absorbing fluids, as fragrance booster, for decreasing skin friction, for modifying skin feel, and/or for modifying the skin appearance for a wet cosmetic composition is provided, wherein the mineral blend comprises a first component being selected from natural ground calcium carbonate and/or precipitated calcium carbonate, and a second component being a surface-reacted calcium carbonate, wherein the surface-reacted calcium carbonate is a reaction product of natural ground calcium carbonate or precipitated calcium carbonate with carbon dioxide and at least one H3O+ ion donor, wherein the carbon dioxide is formed in situ by the at least one H3O+ ion donor treatment and/or is supplied from an external source.

According to a preferred embodiment, the mineral blend is used as fragrance booster for a wet cosmetic composition.

The wet cosmetic composition of the present invention preferably does not contain certain materials or components. According to one embodiment, the wet cosmetic composition is free of talc or a talc-containing material. A “talc or a talc-containing material” are known to the skilled person. “Talc” refers to a clay mineral comprising a hydrated magnesium silicate, which may be described by the chemical formula Mg3Si4010(OH)2. Talc has a value of 1 on the Mohs hardness scale. A “talc-containing material” refers to every material, which contains talc as a component. A “talc-containing material” is, for example, ultramafic rock such as soapstone.

According to another embodiment, the wet cosmetic composition does not contain a silicate- and/or aluminate-containing material. “Silicate- and/or aluminate-containing material” are known to the skilled person and may refer to, for example, mica, kaolin or silica.

The wet cosmetic composition may consist of the mineral blend as described above and water However, in most cases, the wet cosmetic composition according to the present invention may comprise further components in addition to the mineral blend as described herein. The skilled person knows materials, which are allowed and/or suitable for use in a wet cosmetic composition, and will select such materials according to the form of the composition and its application.

The pH value of the composition may be adjusted to any value suitable for a cosmetic composition. For example, the cosmetic composition may have a pH value of ≤8.5, more preferably ≤8.0, even more preferably ≤7.5, still more preferably ≤7.0 and most preferably from 4.0 to 7.0.

According to one embodiment, the wet cosmetic composition comprises a water content of at least 15 wt.-%, preferably at least 20 wt.-%, more preferably at least 25 wt.-%, and most preferably at least 30 wt.-%, based on the total weight of the wet cosmetic composition. The water may be selected from tap water, distilled water, deionized water, or mixtures thereof, and preferably is deionized water.

According to another embodiment, the wet cosmetic care composition further comprises at least one oil. According to a preferred embodiment, the wet cosmetic composition further comprises water and at least one oil. An “oil” in the meaning of the present invention is a liquid or solid silicon- and/or hydrocarbon-containing compound. The at least one oil may be selected from any oil which is suitable to be used in cosmetic compositions. Oils which are suitable for use in cosmetic compositions are known to the skilled person and are described in, for example, Regulation EC No 1223/2009 of the European Parliament and of the Council of 30 Nov. 2009, and must not form part of the list of prohibited substances disclosed therein.

According to one embodiment of the present invention, the at least one oil is selected from the group consisting of vegetable oils and esters thereof, alkanecoconutester, plant extracts, animal fats, siloxanes, fatty acids and esters thereof, petrolatum, glycerides and pegylated derivatives thereof, and mixtures thereof.

For example, a suitable vegetable oil may be palm oil, soybean oil, rapeseed oil, sunflower seed oil, peanut oil, cottonseed oil, palm kernel oil, coconut oil, olive oil, jojoba oil, corn oil, jumbú oil, guava oil, grape seed oi, hazelnut oil, linseed oil, rice bran oil, safflower oil, sesame oil, acai palm oil, graviola oil, tucuma oil, brazil oil, carapa oil, buriti oil, passion fruit oil or pracaxi oil.

Suitable plant extracts may be prepared, for example, from Castanea Sativa, Prunus Dulcis, Juglans Regia L., Olea Europaea, Helichrysum stoechas, Quercus Robur, Glycyrrhiza Glabra, Vitis Vinifera, Crataegus Monogyna Jacq, or Pinus Pinaster.

Suitable animal fats can be obtained, for example, from tallow.

Suitable siloxanes are, for example, dimethicone, cetyl dimethicone, dimethiconol, detearyl methicone, cyclopentasiloxane, cyclomethicone, stearyl dimethicone, trimethylsilylamodimethicone, stearoxy dimethicone, amodimethicone, behenoxy dimethicone, dimethicone copolyol, polysiloxane, laurylmethicone copolyol or cetyl dimethicone copolyol.

Suitable fatty acids are, for example, palmitic acid, stearic acid, myristic acid, oleic acid, palmitoleic acid, linoleic acid, linolenic acid, capric acid, caprylic acid, arachidonic acid and esters thereof.

Suitable petrolatum may be any petrolatum with a refined grade approved for cosmetic use, and preferably has a melting point between 35° C. and 70° C.

Suitable glycerides are, for example, mono-, di, or triglycerides from palmitic acid, stearic acid, myristic acid, oleic acid, palmitoleic acid, linoleic acid, linolenic acid, capric acid, caprylic acid, and mixtures thereof.

In one embodiment, the at least one oil comprises, preferably consists of, one oil. Alternatively, the at least one oil comprises, preferably consists of, two or more oils. For example, the at least one oil comprises, preferably consists of, two or three oils. Preferably, the at least one oil comprises, preferably consists of, two or more oils.

It is appreciated that the wet cosmetic composition may comprise the water and/or the at least one oil and their amounts in dependence of the wet cosmetic composition to be prepared and/or the manufacturer's needs. According to one embodiment, the water is present in an amount of from 15 to 95 wt.-%, preferably from 20 to 90 wt.-%, more preferably from 25 to 80 wt.-%, even more preferably from 35 to 75 wt.-%, and most preferably from 45 to 65 wt.-%, based on the total weight of the wet cosmetic composition. According to another embodiment, the at least one oil is present in an amount of from 1 to 95 wt.-%, preferably from 2 to 75 wt.-%, more preferably from 5 to 55 wt.-%, even more preferably from 7.5 to 35 wt.-%, and most preferably from 10 to 20 wt.-%, based on the total weight of the wet cosmetic composition.

In case the wet cosmetic composition comprises water and at least one oil, the composition may be a water-based dispersion or an oil-based dispersion. Thus, according to one embodiment, the wet cosmetic composition is a water-based dispersion. According to another embodiment, the wet cosmetic composition is an oil-based dispersion. According to a preferred embodiment, the wet cosmetic composition is a water-based dispersion. A “water-based dispersion” in the meaning of the present invention refers to a composition wherein water forms a continuous phase and the oil a dispersed phase, i.e. the oil is dispersed in the continuous water phase. An “oil-based dispersion” in the meaning of the present invention refers to a composition wherein oil forms a continuous phase and water a dispersed phase, i.e. water is dispersed in the continuous water phase. According to yet another embodiment, the water is present in an amount of from 15 to 95 wt.-%, preferably from 20 to 90 wt.-%, more preferably from 25 to 80 wt.-%, even more preferably from 35 to 75 wt.-%, and most preferably from 45 to 65 wt.-%, and the at least one oil is present in an amount of from 1 to 95 wt.-%, preferably from 2 to 75 wt.-%, more preferably from 5 to 55 wt.-%, even more preferably from 7.5 to 35 wt.-%, and most preferably from 10 to 20 wt.-%, based on the total weight of the wet cosmetic composition.

As described above, the intra and interpore structure of the surface-reacted calcium carbonate can make it a superior agent to deliver previously adsorbed and/or absorbed materials over time relative to common materials having similar specific surface areas. Thus, generally, any agent fitting into the intra- and/or inter particle pores of the surface-reacted calcium carbonate is suitable to be transported by the surface-reacted calcium carbonate according to the invention. Accordingly, it is possible that the cosmetic and/or skin care composition comprises at least one active agent being adsorbed onto and/or absorbed into the surface of the surface-reacted calcium carbonate. According to one embodiment of the present invention, the wet cosmetic composition comprises at least one active agent being adsorbed onto and/or absorbed into the surface of the surface-reacted calcium carbonate.

According to a preferred embodiment of the present invention, the at least one active agent is selected from pharmaceutically active agents, biologically active agents, vitamins, disinfecting agents, preservatives, flavouring agents, surfactants, oils, fragrances, essential oils such as limonene or mint oil, and mixtures thereof, and preferably biologically active agents, scented oils and essential oils.

The at least one active agent may be adsorbed onto and/or absorbed into the surface of the surface-reacted calcium carbonate in specific amounts. According to one embodiment of the present invention, the amount of the at least one agent being adsorbed onto and/or absorbed into the surface of the surface-reacted calcium carbonate ranges from 0.1 to 99 wt.-%, based on the weight of the surface-reacted calcium carbonate, preferably ranges from 30 to 95 wt.-%, more preferably from 50 to 90 wt.-%, and most preferably from 70 to 85 wt.-%.

The wet cosmetic composition may also comprise further additives. Additives that are suitable for wet cosmetic compositions are known to the skilled person and are described in, for example, Regulation EC No 1223/2009 of the European Parliament and of the Council of 30 Nov. 2009, and must not form part of the list of prohibited substances disclosed therein. According to one embodiment of the present invention, the wet cosmetic composition further comprises at least one additive selected from the group consisting of bleaching agents, thickeners, stabilizers, chelating agents, preserving agents, wetting agents, emulsifiers, emollients, fragrances, colorants, skin tanning compounds, antioxidants, minerals, pigments, UV-A and/or UV-B filter, and mixtures thereof. According to one embodiment, the wet cosmetic composition comprises further additives in an amount of from 0.1 to 40 wt.-%, preferably from 0.1 to 35 wt. %, more preferably from 10 to 35 wt.-%, based on the total weight of the wet cosmetic composition.

For example, the emulsifier can be an ionic emulsifier, more preferably and anionic or cationic emulsifier. The emulsifier can be of natural vegetable origin e.g. polyglycerol ester or synthetic. More preferably, the emulsifier may be selected from the group comprising PEG compounds, PEG-free emulsifier, silicone-based emulsifier, silicones, waxes and mixtures thereof. For example, the emulsifier may be selected from the group comprising PEG compounds such as PEG-8 myristate, PEG-30 glyceryl cocoate, PEG-80 glyceryl cocoate, PEG-15 soyamide/IPDI copolymer, PEG-40 sorbitan peroleate, PEG-150 stearate and mixtures thereof, carbomer, carboxymethylcellulose, ceresin (aka mineral wax), diethanolamine (DEA), isopropyl stearate, isopropyl laurate, isopropyl palmitate, isopropyl oleate, polysorbate 20, polysorbate 60, polysorbate 80, propylene glycol, sorbitan stearate, sorbitan laurate, sorbitan palmitate, sorbitan oleate, steareth-20, triethanolamine (TEA), beeswax, candelilla wax, carnauba wax, cetearyl alcohol, cetearyl wheat bran glycosides, cetearyl wheat straw glycosides, decyl glucoside, jojoba, lecithin, vegetable glycerin, xanthan gum, coco glucoside, coconut alcohol, arachidyl alcohol, behenyl alcohol, arachidyl glucoside, and mixtures thereof.

The fragrance may be selected from a natural and/or synthetic fragrance known as being suitable in cosmetic formulations.

The colorant may be selected from a natural and/or synthetic colorant, pigment or dye such as Fe2O3, ZnO, TiO2, mica, talc, bismuth oxychloride, and mixtures thereof.

According to one embodiment, the skin tanning compound is preferably dihydroxyacetone (DHA) and/or erythrulose. For example, the skin tanning compound may be dihydroxyacetone (DHA) or erythrulose. Alternatively, the skin tanning compound may be dihydroxyacetone (DHA) in combination with erythrulose.

According to one embodiment, the wet cosmetic composition further comprises at least one emollient. Examples of suitable emollients are isocetylstearoylstearate, ethylhexyl stearate, octyldodecyl stearoyl stearate, isocetyl stearate, isopropyl isostearate, isostearyl isostearate, ethylhexyl hydroxystearate, ethylhexyl palmitate, isopropyl palmitate, neopentyl glycol diheptanoate, ethylhexyl isononanoate, isononyl isononanoate, cetearyl isononanoate, cetearyl octanoate, diisopropyl adipate, dicapryl adipate, diisostearylmalate, decyl oleate, isodecyl oleate, diisopropyl myristate, isostearyl neopentanoate, octyl dodecyl neopentanoate, ethylhexyl cocoate, PEG-7 glyceril cocoate, C12-15 alkyl benzoate, C16-17 alkyl benzoate, stearyl benzoate, isostearyl benzoate, ethylhexyl benzoate, octyldodecyl benzoate, cocoglyceride, coconut alkanes, coco-caprylate/caprate, and mixtures thereof. For example, the cosmetic composition may further comprise a mixture of cocoglyceride, isononyl isononanoate, coconut alkanes and coco-caprylate/caprate as emollient.

Additionally or alternatively, the wet cosmetic composition further comprises at least one thickener. Examples of suitable thickener for a water-based dispersion are thickener based on silicate such as magnesium silicate, aluminium silicate and mixtures thereof, hydroxyethyl cellulose, cellulose, microcrystalline cellulose, xanthan gum or polyacrylamide. Examples of suitable thickener for an oil-based dispersion are selected from the group comprising silicate such as magnesium silicate, aluminium silicate, silica dimethylsilicate, hydrophobic fumed silica, polyacrylic acid, salts of polyacrylic acid, derivatives of polyacrylic acid, PEG compounds such as PEG-8 myristate, PEG-30 glyceryl cocoate, PEG-80 glyceryl cocoate, PEG-15 soyamide/IPDI copolymer, PEG-40 sorbitan peroleate, PEG-150 stearate and mixtures thereof, methyl cellulose, ethyl cellulose, propyl cellulose, carboxymethylcellulose, xanthan gum, ammonium acryloyldimethyltaurate/VP copolymer and mixtures thereof.

Additionally or alternatively, the wet cosmetic composition further comprises at least one preserving agent. Examples of suitable preserving agents are phenoxyethanol, ethylhexylglycerin, parabens such as methyl paraben, ethyl paraben, propyl paraben, butyl paraben, isobutyl paraben and mixtures thereof, para-oxybenzoic acid alkyl ester, benzoic acid, sodium benzoate, sorbic acid, potassium sorbate, salicylic acid, carbolic acid, sorbic acid, hexachlorophene, benzalkonium chloride, chlorhexidine chloride, trichlorocarbanilide, and mixtures thereof, or plant extracts with preservative function such as rosemary extracts. For example, said mixture may comprise phenoxyethanol, methyl paraben, ethyl paraben and isobutyl paraben.

Examples of suitable chelating agents are a polyphosphate, ethylenediamine-N,N,N′,N′-tetraacetic acid (EDTA), pyridine-2,6-dicarboxylic acid (DPA), diethylenetriaminepentaacetic acid (DTPA), N,N-bis(carboxymethyl)glycine (NTA), ammonium diethyldithiophosphate (DDPA), disodium ethylenediamine-tetraacetate (Na2H2EDTA), calcium-disodium-ethylenediamine-tetraacetate (CaNa2EDTA), citric acid and salts of citric acid, sodium gluconate, and mixtures thereof.

Examples of suitable wetting agents are primary alcohols such as 1-ethanol, 1-propanol, 1-butanol, isobutanol 1-pentanol, isoamyl alcohol, 2-methyl-1butanol, 1-hexanol,1-heptanol, 1-octanol, 1-nonanol, 1-decanol, 1-undecanol, 1-dodecanol, 1-tridecanol, 1-tetradecanol, 1-pentadecanol, cetyl alcohol, 1-heptadecanol, stearyl alcohol, 1-nonadecanol and mixtures thereof, secondary alcohols such as isopropanol, 2-butanol, 2-pentanol, 2-hexanol, 2-heptanol and mixtures thereof, tertiary alcohols such as tert.-butyl alcohol, tert.-amyl alcohol, 2-methyl-2-pentanol, 2-methylhexan-2-ol, 2-methylheptan-2-ol, 3-methyl pentanol, 3-methyloctan-3-ol and mixtures thereof, diols such as 1,2-diols or 1,3-diols, e.g. 1,3-propandiol, urea, and mixtures thereof.

Examples of suitable antioxidants are butylhydroxyanisol (BHA), butylhydroxytoluol (BHT), gallate, carotinoid, polyphenols such as resveratrol, flavonoid and mixtures thereof, derivatives of polyphenols, ascorbic acid and salts thereof, tocopherol and salts thereof, betacarotin, ubichinon, tocotrienol, dihydroquercetin, antioxidants of natural origin, and mixtures thereof.

Examples of suitable pigments are inorganic red pigments such as iron oxide, ferric hydroxide and iron titanate, inorganic brown pigments such as γ-iron oxide, inorganic yellow pigments such as yellow iron oxide and yellow ocher, inorganic black pigments such as black iron oxide and carbon black, inorganic purple pigments such as manganese violet and cobalt violet, inorganic green pigments such as chromium hydroxide, chrome oxide, cobalt oxide and cobalt titanate, inorganic blue pigments such as iron blue and ultramarine, particulate powders such as particulate titanium oxide, particulate cerium oxide and particulate zinc oxide, laked tar dyes, laked natural dyes, and synthetic resin powders combining foregoing powders.

The bleaching agent may be selected from one or more of a vitamin B3 compound or its derivative e.g. niacin, nicotinic acid or niacinamide or other well-known bleaching agents e.g. adapalene, aloe extract, ammonium lactate, anethole derivatives, apple extract, arbutin, azelaic acid, kojic acid, bamboo extract, bearberry extract, bletilla tuber, bupleurum falcatum extract, burnet extract, butyl hydroxy anisole, butyl hydroxy toluene, citrate esters, Chuanxiong, Dang-Gui, deoxyarbutin, 1,3-diphenyl propane derivatives, 2,5-dihydroxybenzoic acid and its derivatives, 2-(4-acetoxyphenyl)-1,3-dithane, 2-(4-hydroxyphenyl)-1,3-dithane, ellagic acid, escinol, estragole derivatives, Fadeout (Pentapharm), Fangfeng, fennel extract, ganoderma extract, gaoben, Gatuline Whitening (Gattlefosse), genistic acid and its derivatives, glabridin and its derivatives, gluco pyranosyl-1-ascorbate, gluconic acid, glycolic acid, green tea extract, 4-hydroxy-5-methyl-3[2H]-furanone, hydroquinone, 4-hydroxyanisole and its derivatives, 4-hydroxy benzoic acid derivatives, hydroxycaprylic acid, inositol ascorbate, lemon extract, linoleic acid, magnesium ascorbyl phosphate, Melawhite (Pentapharm), moms alba extract, mulberry root extract, 5-octanoyl salicylic acid, parsley extract, phellinus linteus extract, pyrogallol derivatives, 2,4-resorcinol derivatives, 3,5-resorcinol derivatives, rose fruit extract, salicylic acid, Song-Yi extract, 3,4,5-trihydroxybenzyl derivatives, tranexamic acid, vitamins like vitamin B6, vitamin B12, vitamin C, vitamin A, dicarboxylic acids, resorcinol derivatives, extracts from plants viz. rubia and symplocos, hydroxycarboxylic acids like lactic acid and their salts e.g. sodium lactate, and mixtures thereof. Vitamin B3 compound or its derivative e.g. niacin, nicotinic acid or niacinamide are the more preferred bleaching agents, most preferred being niacinamide. Niacinamide, when used, is preferably present in an amount in the range of 0.1 to 10 wt.-%, more preferably 0.2 to 5 wt.-%, based on the total weight of the wet cosmetic composition.

UV-A and/or UV-B filter may be selected from inorganic UV filter and/or organic UV filter. Suitable inorganic UV filter are, for example, selected from the group consisting of titanium dioxide, zinc oxide, iron oxide, hydroxyapatite, cerium oxide, calcium-doped cerium oxide, cerium phosphate, and mixtures thereof. Suitable organic UV filter are, for example, selected from the group comprising cinnamic acid and its salts, derivatives of salicylic acid and its salts, benzophenones, derivatives of aminobenzoic acid and its salts, dibenzoylmethanes, benzylidenecamphor derivatives, benzimidazole derivatives, diphenylacrylate derivatives, acrylamide derivatives, benzotriazole derivatives, triazine derivatives, benzalmalonate derivatives, aminobenzoate derivatives, octocrylene, and mixtures thereof.

It is appreciated that the wet cosmetic composition may comprise the at least one further additive and its amount in dependence of the wet cosmetic composition to be prepared and/or the manufacturer's needs. For example, the wet cosmetic composition may comprise 0.1 to 10 wt.-% of thickeners, stabilizers, chelating agents, bleaching agents, wetting agents, emulsifiers, emollients, and/or skin tanning compounds, and/or 0.1 to 15 wt.-% of preserving agents, fragrances, colorants, antioxidants, minerals, pigments, UV-A and/or UV-B filter wherein the wt.-% is based on the total weight of the cosmetic composition.

In one embodiment, the at least one additive comprises, preferably consists of, one additive. Alternatively, the at least one additive comprises, preferably consists of, two or more additives. For example, the at least one additive comprises, preferably consists of, ten to fifteen additives. Preferably, the at least one additive comprises, preferably consists of, two or more additives.

Furthermore, the wet cosmetic composition may have a certain Brookfield viscosity. For the purpose of the present invention, the term “viscosity” or “Brookfield viscosity” refers to Brookfield viscosity. The Brookfield viscosity is for this purpose measured by a Brookfield (Typ RVT) viscometer at 25° C.±1° C. at 100 rpm after 30 seconds using an appropriate spindle and is specified in mPas. According to one embodiment of the present invention, the cosmetic and/or skin care composition has a Brookfield viscosity in a range from 4 000 to 50 000, preferably from 10 000 to 45 000, more preferably from 15 000 to 40 000, even more preferably from 20 000 to 40 000, and most preferably from 25 000 to 40 000 mPas at 25° C.

According to an exemplary embodiment, a wet cosmetic composition is provided, wherein

the first component is a natural ground calcium carbonate having a volume median particle size d₅₀ from 0.8 to 8 μm, and a specific surface area from 1 to 20 m²/g,

the second component is a surface-reacted calcium carbonate having a volume median particle size d₅₀ from 3 to 10 μm, and a specific surface area from 30 to 90 m²/g, wherein the surface-reacted calcium carbonate is a reaction product of natural ground calcium carbonate or precipitated calcium carbonate with carbon dioxide and at least one H₃O⁺ ion donor, wherein the carbon dioxide is formed in situ by the at least one H₃O⁺ ion donor treatment and/or is supplied from an external source, and the wet cosmetic product is a skin care product or a deodorant.

Method of Producing the Mineral Blend

The mineral blend may be produced by any process known in the art. According to one embodiment, a process for preparing the mineral blend comprises the following steps:

a) providing a first component being a natural ground calcium carbonate and/or a precipitated calcium carbonate,

b) providing a second component being a surface-reacted calcium carbonate, wherein the surface-reacted calcium carbonate is a reaction product of natural ground calcium carbonate or precipitated calcium carbonate with carbon dioxide and at least one H₃O⁺ ion donor, wherein the carbon dioxide is formed in situ by the at least one H₃O⁺ ion donor treatment and/or is supplied from an external source, and

c) mixing the first component of step a) with the second component of step b).

The first component of step a) may be provided in dry form or in form of an aqueous suspension. According to one embodiment, the first component of step a) is provided in dry form. Preferably, the first component provided in step a) has a water content of below 3.0 wt.-%, preferably of below 2.0 wt.-%, and more preferably of below 1.0 wt.-%, based on the total dry weight of the first component. The residual water content may be determined as described above for the wet cosmetic composition.

According to another embodiment, the first component of step a) is provided in form of an aqueous suspension, preferably in form of a slurry. Preferably, the first component provided in step a) is present in the slurry of from 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

Similarly, the second component provided in step b) may also be provided in dry form or in form of an aqueous suspension. According to one embodiment, the second component of step b) is provided in dry form. Preferably, the second component has a water content of below 10 wt.-%, preferably of below 5.0 wt.-%, and more preferably of below 3.0 wt.-%, based on the total dry weight of the second component. The residual water content may be determined as described above for the wet cosmetic composition.

In an alternative embodiment, the second component of step b) is provided in form of an aqueous suspension, preferably in form of a slurry. Preferably, the second component provided in step b) is present in the slurry of from 1 wt.-% to 90 wt.-%, more preferably 3 wt.-% to 60 wt.-%, even more preferably 5 wt.-% to 40 wt.-%, and most preferably 10 wt.-% to 25 wt.-%, based on the weight of the slurry.

The first component of step a) and the second component of step b) may be provided in dry form. Thus, according to one embodiment, the first component of step a) and the second component of step b) are provided in dry form.

However, it is also possible that the first component of step a) and/or the second component of step b) is provided in form of an aqueous suspension. Thus, according to one embodiment, the first component of step a) is provided in dry form, and the second component of step b) is provided in form of an aqueous suspension, preferably in form of a slurry. According to another embodiment, the first component of step a) is provided in form of an aqueous suspension, preferably in form of a slurry, and the second component of step b) is provided in dry form. According to yet another embodiment, the first component of step a) and the second component of step b) are provided in form of an aqueous suspension, preferably in form of a slurry. According to one embodiment, an aqueous dispersion, preferably a slurry, is obtained in mixing step c) having a solids content of from 1 to 90 wt.-%, preferably from 3 to 60 wt.-%, more preferably from 10 to 45 wt.-%, and most preferably from 10 to 40 wt.-%, based on the total weight of the aqueous dispersion obtained in mixing step c).

In case the first component of step a) and the second component of step b) are provided in dry form, mixing step c) may be a dry blending step. According to one embodiment, mixing step c) is a dry blending step. Preferably, the dry blending step is carried out with a plow share mixer, a ribbon mixer or a cone single shaft mixer, and more preferably with a cone single shaft mixer. The skilled person is familiar with such type of mixers.

The first component of step a) and the second component of step b) may be added to a mixing vessel in mixing step c), simultaneously or in any order. According to one embodiment, the first component of step a) is added to the second component of step b) to a mixing vessel in mixing step c), or vice versa. According to another embodiment, the first component of step a) and the second component of step b) are added simultaneously to a mixing vessel in mixing step c).

In case the first component of step a) and/or the second component of step b) is provided in form of an aqueous suspension, mixing step c) may be a wet mixing step. According to one embodiment, mixing step c) is a wet mixing step. A “wet mixing step” in the meaning of the present invention refers to the mixing of an aqueous suspension or a slurry. The wet mixing step may be carried out under conventional mixing conditions. The skilled man will adapt these mixing conditions (such as the configuration of mixing pallets and mixing speed) according to his process equipment. It is appreciated that any wet mixing method which would be suitable to a cosmetic composition may be used.

If mixing step c) is a wet mixing step, the process may further comprises a drying step d) for providing a dry mineral blend. The drying step d) may be carried out by any drying process, which is suitable for drying the mineral blend. Preferably, the drying step d) is a spray drying step. According to one embodiment, the process further comprises a drying step d), preferably a spray drying step or a superheated steam drying step, and more preferably a spray drying step. Preferably, the spray drying step is carried out at between 80 and 250° C., more preferably between 100 and 200° C., until a constant dry weight of the mineral blend is reached. Drying particulate matter by superheated steam drying is known to the skilled person. For example, the superheated steam drying step may be carried out as described in WO2012140028.

Method of Producing the Wet Cosmetic Composition

A method for the preparation of the wet cosmetic composition comprises at least the provision of the mineral blend as a cosmetic agent.

The mineral blend may be provided in any suitable liquid or dry form. For example, the mineral blend may be in form of a powder and/or a suspension. The suspension can be obtained by mixing the mineral blend with a solvent, preferably water. The mineral blend to be mixed with a solvent, and preferably water, may be provided in any form, for example, as suspension, slurry, dispersion, paste, powder, a moist filter cake or in pressed or granulated form, and preferably is provided as a powder.

The term “dispersion” or “suspension” in the meaning of the present invention refers to a system comprising a dispersing medium or solvent and at least one inorganic particulate material, wherein at least a part of the particles of the at least one inorganic particulate material are present as insoluble solids or suspended particles in the dispersing medium or solvent. The suspension can be undispersed or dispersed, i.e. the suspension includes a dispersant, and thus, forms a dispersion, e.g. an aqueous dispersion. Suitable dispersants are known in the art, and may be selected, e.g., from polyelectrolytes, polyhydroxystearic acid, acetylacetone, propylamine, oleic acid, polyacrylates, carboxymethylcellulose based dispersants, and mixtures thereof. The solids content of the suspension, preferably aqueous suspension, of the mineral blend may be from 1 to 85 wt.-%, more preferably from 5 to 75 wt.-%, and most preferably from 10 to 40 wt.-%, based on the total weight of the suspension.

In case the mineral blend is provided in dry form, the water content of the mineral blend can be between 0.01 and 5 wt.-%, based on the total weight of the mineral blend. The water content of the mineral blend can be, for example, less than or equal to 1.0 wt.-%, based on the total weight of the mineral blend, preferably less than or equal to 0.5 wt.-%, and more preferably less than or equal to 0.2 wt.-%. According to another example, the water content of the mineral blend may be between 0.01 and 0.15 wt.-%, preferably between 0.02 and 0.10 wt.-%, and more preferably between 0.03 and 0.07 wt.-%, based on the total weight of the mineral blend.

A method for the preparation of the wet cosmetic composition may further comprise the provision of water and, optionally, least one oil and the mixing of the water and the optional at least one oil with the mineral blend.

The mixing of the water and/or the at least one oil and the mineral blend may be carried out in any manner known by the skilled person. The mixing may be carried out under conventional mixing conditions. The skilled man will adapt these mixing conditions (such as the configuration of mixing pallets and mixing speed) according to his process equipment. It is appreciated that any mixing method which would be suitable to form a wet cosmetic composition may be used.

In case, the method further comprises the provision of water and at least one oil, the mixing may be carried out in any order. Preferably, the water and the at least one oil are combined and mixed to form a mixture followed by the addition and mixing of the mineral blend.

Mixing can be carried out at temperatures typically used for preparing a cosmetic base formulation. Preferably, mixing is carried out at a temperature in the range from 15 to 100° C., more preferably from 20 to 85° C. such as of about 45° C.

The method for the preparation of the wet cosmetic composition may further comprise the provision of at least one additive. The combining and mixing of the at least one additive and the mineral blend may also be carried out under conventional mixing conditions. The skilled man will adapt these mixing conditions (such as the configuration of mixing pallets and mixing speed) according to his process equipment. It is appreciated that any mixing method which would be suitable to form a cosmetic composition may be used.

In case, the method comprises the provision of the mineral blend, water and/or at least one oil, and at least one additive, and preferably two or more additives, the combining and mixing may be carried out in any order.

The scope and interest of the invention will be better understood based on the following examples which are intended to illustrate certain embodiments of the present invention and are non-limitative.

### EXAMPLES

### 1. Measurement Methods

In the following, measurement methods implemented in the examples are described.

Particle Size Distribution

Volume determined median particle size d50 (vol) and the volume determined top cut particle size d98 (vol) was evaluated using a Malvern Mastersizer 3000 Laser Diffraction System (Malvern Instruments Plc., Great Britain). The d50 (vol) or d98 (vol) value indicates a diameter value such that 50% or 98% by volume, respectively, of the particles have a diameter of less than this value. The raw data obtained by the measurement was analyzed using the Mie theory, with a particle refractive index of 1.57 and an absorption index of 0.005. The methods and instruments are known to the skilled person and are commonly used to determine particle size distributions of fillers and pigments. The measurement was carried out in an aqueous solution of 0.1 wt.-% Na4P2O7. The samples were dispersed using a high-speed stirrer and supersonicated.

Specific Surface Area (SSA)

The specific surface area was measured via the BET method according to ISO 9277:2010 using nitrogen, following conditioning of the sample by heating at 250° C. for a period of 30 minutes. Prior to such measurements, the sample was filtered within a Buchner funnel, rinsed with deionised water and dried at 110° C. in an oven for at least 12 hours.

Intra-Particle Intruded Specific Pore Volume (in Cm3/g)

The specific pore volume was measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step was 20 seconds. The sample material was sealed in a 5 cm3 chamber powder penetrometer for analysis. The data were corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine inter-particle packing of the particles themselves. If they also have intra-particle pores, then this region appears bi-modal, and by taking the specific pore volume intruded by mercury into pores finer than the modal turning point, i.e. finer than the bi-modal point of inflection, the specific intra-particle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

Sensorial Analysis

A protocol of sensory evaluation was developed to characterize creams and deodorants.

The sensory properties were tested by applying cream or deodorant, respectively, on the hand or using the finger. To evaluate sensorial parameters like slipperiness the samples have been taken using the finger while spreadbility has been ranked by putting the samples on the hand. The samples were then rated on a 0-10 scale with 0 representing no opacity, for example, and 10 being the most severe degree of affected parameter.

The following attributes were thus identified and defined:

Visual Attributes (Appearance):
Opacity (product's colour as it is): transparent to opaque

Attributes Evaluated During the Application:
Slipperiness (ease of distribution the product over the application finger): low-high (high desired)
Spreadability (ease of distribution the product over the skin): low-high (high desired)

Attributes Evaluated after the Application (after-Feel):
Greasiness (oily aspect on the skin): low-high (low desired)
Stickiness (residual sticky film on skin): low-high (low desired)
Softness (softness of skin after of application of composition): low-high (high desired)

Coating of Contrast Cards

Contrast cards were coated by using the respective coating compositions and applying them with a coater gap of 50 μm on the surface of the contrast card. The contrast cards used are Leneta contrast cards, form 3-B-H, size 7-5/8×11-3/8 (194×289 mm), sold by the company Leneta, and distributed by Novamart, Stafa, Switzerland.

Determination of Colour Values (Rx, Ry, Rz)

The colour values Rx, Ry, Rz are determined over the white and black fields of the Leneta contrast card, and are measured with a spectraflas SF 450 X spectrophotometer of the company Datacolor, Montreuil, France.

Contrast Ratio (Opacity) of the Surface of a Coated Contrast Card

Contrast ratio values are determined according to ISO 2814 at a spreading rate of approx. 20 m²/l.

The contrast ratio is calculated as described by the equation below:

$$Contrast ratio [ \% ] = \frac{{Ry}_{black}}{{Ry}_{white}} \times 100 \%$$

with Ry_{black} and Ry_{white} being obtained by the measurement of the color values.

Determination of the Covering Power of Base Compositions

In order to determine the covering power (coverage) of the mineral blend and talc, respectively, base compositions comprising different concentrations of said materials, namely 5 and 10 wt.-% were prepared. The covering power of the respective base compositions was determined by measuring the colour values (Rx, Ry, Rz) and then calculating the contrast ratio, as described above. The ingredients of the base formulation and the base composition comprising the mineral blend or talc, respectively, are compiled in Tables 1 and 2 below.

The base composition was prepared as follows:

The demineralized water was added to a beaker, then, Calgon, Bermocoll and the sodium hydroxide solution were added under stirring with a lab dissolver until all ingredients were dissolved. Then the other ingredients listed in Table 1 up to Byk 349 were added while continuously stirring the mixture. Then the demineralized water was added and the resulting mixture was thoroughly mixed. Finally, the binding agent Mowilith was added during continuous stirring of the mixture at a speed of 100 rpm to obtain the final base formulation.

This base formulation was used for the preparation of base compositions with different pigment concentrations, as listed in Table 2 below.

TABLE 1
Ingredients of base formulation (wt.- % are based
on total amount of base formulation).

| Ingredients | wt.- % |
|---|---|
| Demineralized water | 40.0 |
| Calgon N new | 0.2 |
| Bermocoll EHM 200 | 1.0 |
| Sodium hydroxide solution, 10% | 0.6 |
| Byk 011 | 2.0 |
| Texanol | 0.5 |
| Butyldiglycol acetate | 0.5 |
| Dowanol ™ DPnB | 1.0 |
| Byk 019 | 0.5 |
| Coapur ™ 2025 | 1.8 |
| Mergal 723 K | 0.2 |
| Demineralized water | 5.0 |
| Ecodis ™ P 90 | 0.6 |
| Disperbyk ®-181 | 1.0 |
| Byk 349 | 0.4 |
| Demineralized water | 14.7 |
| Mowilith ® DM 2425, 50% | 30.0 |
| Total | 100.0 |

TABLE 2
Base compositions (wt.- % are based on
total amount of base composition).

| | Composition No. | | | |
|---|---|---|---|---|
| | A-1 | A-2 | | |
| | (comparative) | (comparative.) | A-3 | A-4 |
| Ingredients | wt.- % | wt.- % | wt.- % | wt.- % |
| Base | 95.0 | 90.0 | 95.0 | 90.0 |
| composition | | | | |
| of Table 1 | | | | |
| Mineral blend | — | — | 5.0 | 10.0 |
| Talc | 5.0 | 10.0 | — | — |

### 2. Materials

Natural Ground Calcium Carbonate (NGCC)

A high purity natural calcium carbonate having a d50 (vol) of 3.0 μm and a SSA of 2.5 m2/g that is commercially available from Omya.

Surface-Reacted Calcium Carbonate (SRCC)

Surface-reacted calcium carbonate (SRCC) (d50 (vol)=6.6 μm, d98=13.7 μm, SSA=59.9 m2/g). The intra-particle intruded specific pore volume is 0.939 cm3/g (for the pore diameter range of 0.004 to 0.51 μm).

SRCC was obtained by preparing 350 litres of an aqueous suspension of ground calcium carbonate in a mixing vessel by adjusting the solids content of a ground limestone calcium carbonate from Omya SAS, Orgon having a weight based median particle size d50 (wt) of 1.3 μm, as determined by sedimentation, such that a solids content of 10 wt.-%, based on the total weight of the aqueous suspension, is obtained. Whilst mixing the slurry at a speed of 6.2 m/s, 11.2 kg phosphoric acid was added in form of an aqueous solution containing 30 wt.-% phosphoric acid to said suspension over a period of 20 minutes at a temperature of 70° C. After the addition of the acid, the slurry was stirred for additional 5 minutes, before removing it from the vessel and drying using a jet-dryer.

Talc

Microtalc Pharma 8 having a d50 (vol) of 2.2 μm, a d98 (vol) of 9 μm and an SSA of 12 m2/g that is commercially available from Elementis.

Mineral Blend

The mineral blend was composed of 90 wt.-% NGCC and 10 wt.-% SRCC, based on the total weight of the mineral blend.

### 3. Examples

### Example 1—Skin Cream Composition

Skin cream compositions were prepared as follows:
Heat phase A and B separately at 80° C.
Add phase B to phase A while stirring (Heidolph, Faust, 300 rpm)
Cool down at room temperature
Add phase C and D and homogenize (Ultra Turrax T25-D, IKA, 24 000 rpm)
Adjust the pH at 6.0 using lactic acid (10%-solution).

The compositions of the prepared skin creams are compiled in Table 3 below.

TABLE 3
Skin cream compositions (wt.-% values are based on total weight
of the skin cream composition, comp.: comparative).

| | | | B-1 | B-2 | | B-4 | B-5 |
|---|---|---|---|---|---|---|---|
| | Composition No. | INCI | (comp.) | (comp.) | B-3 | (comp.) | (comp.) |
| Phase | Ingredients | Nomenclature | wt.-% | wt.-% | wt.-% | wt.-% | wt.-% |
| A) | Lanette O | Cetearyl Alcohol | 2.00 | 2.00 | 2.00 | 2.00 | 2.00 |
| | Emulium | Tribehenin PEG- | 3.00 | 3.00 | 3.00 | 3.00 | 3.00 |
| | 22MB | 20 Esters | | | | | |
| | Almond Oil | Prunus Amygdalus | 2.00 | 2.00 | 2.00 | 2.00 | 2.00 |
| | | Dulcis | | | | | |
| | | (Almond) Oil | | | | | |
| | Macadamia Oil | Macadamia Ternifolia | 5.00 | 5.00 | 5.00 | 5.00 | 5.00 |
| | | Seed Oil | | | | | |
| | Miglyol 812 | Caprilyc/Capric | 2.00 | 2.00 | 2.00 | 2.00 | 2.00 |
| | | Triglyceride | | | | | |
| | MOD | Octyldodecyl | 4.00 | 4.00 | 4.00 | 4.00 | 4.00 |
| | | Myristate | | | | | |
| | Copherol 1250C | Tocopheryl Acetate | 1.00 | 1.00 | 1.00 | 1.00 | 1.00 |
| B) | Water dem. | Aqua (water) | add. 100 | add. 100 | add. 100 | add. 100 | add. 100 |
| | Propanediol Zemea | Propanediol | 5.00 | 5.00 | 5.00 | 5.00 | 5.00 |
| | Glycerin | Glycerin | 3.00 | 3.00 | 3.00 | 3.00 | 3.00 |
| | Xanthan Gum | Xanthan Gum | 0.10 | 0.10 | 0.10 | 0.10 | 0.10 |
| | Sodium Chloride | Sodium Chloride | 0.50 | 0.50 | 0.50 | 0.50 | 0.50 |
| | Allantoin EP | Allantoin | 0.10 | 0.10 | 0.10 | 0.10 | 0.10 |
| C) | Mineral blend | | — | — | 5.00 | — | — |
| | Talc | | — | 5.00 | — | — | — |
| | SRCC | | — | — | — | 5.00 | — |
| | NGCC | | — | — | — | — | 5.00 |
| D) | Perfume | Perfume | q.s | q.s | q.s | q.s | q.s |
| | Leucidal | Leuconostoc | 3.00 | 3.00 | 3.00 | 3.00 | 3.00 |
| | | Radish Root | | | | | |
| | | Ferment Filtrate | | | | | |
| | | (and) Aqua | | | | | |
| | | | 100.00 | 100.00 | 100.00 | 100.00 | 100.00 |

### Example 2—Deodorant Composition

Deodorant compositions were prepared as follows:
Melt phase A at 75-80° C.
Add part B and homogenize (Ultra Turrax T25-D, IKA, 24 000 rpm)
Melt phase A&B at 75-80° C.
Stir the mixture until cool to about 40° C.
Add phase C to phases A and B
Poor the mixture into forms

The compositions of the prepared deodorants are compiled in Table 4 below.

TABLE 4
Deodorant compositions (wt.-% values are based on total
weight of the deodorant composition, comp.: comparative).

| | | | C-1 | C-2 | | C-4 | C-5 |
|---|---|---|---|---|---|---|---|
| | Composition No. | INCI | (comp.) | (comp.) | C-3 | (comp.) | (comp.) |
| | Ingredients | Nomenclature | wt.-% | wt.-% | wt.-% | wt.-% | wt.-% |
| A) | Sotfisan 100 | Hydrogenated | 38.50 | 38.50 | 38.50 | 38.50 | 38.50 |
| | | Coco-Glycerides | | | | | |
| | Miglyol 812 | Caprylic/Capric | 20.50 | 20.50 | 20.50 | 20.50 | 20.50 |
| | | Triglycerides | | | | | |
| | Tegin | Glyceryl Stearate | 10 | 10 | 10 | 10 | 10 |
| | | SE | | | | | |
| | Beeswax | Cera Alba | 18.50 | 18.50 | 18.50 | 18.50 | 18.50 |
| | Glycerin | Glycerin | 5.00 | 5.00 | 5.00 | 5.00 | 5.00 |
| | Propanediol Zemea | Propanediol | 5.00 | 5.00 | 5.00 | 5.00 | 5.00 |
| B) | Mineral blend | | — | — | 5.00 | — | — |
| | Talc | | — | 5.00 | — | — | — |
| | SRCC | | — | — | — | 5.00 | — |
| | NGCC | | — | — | — | — | 5.00 |
| C) | Lemon Essential | Citrus Limon | 0.40 | 0.40 | 0.40 | 0.40 | 0.40 |
| | Oil | Peel Oil | | | | | |
| | Lavender Essential | Lavandula | 0.40 | 0.40 | 0.40 | 0.40 | 0.40 |
| | Oil | Angustifolia Oil | | | | | |
| | | | 100.00 | 100.00 | 100.00 | 100.00 | 100.00 |

### 4. Results

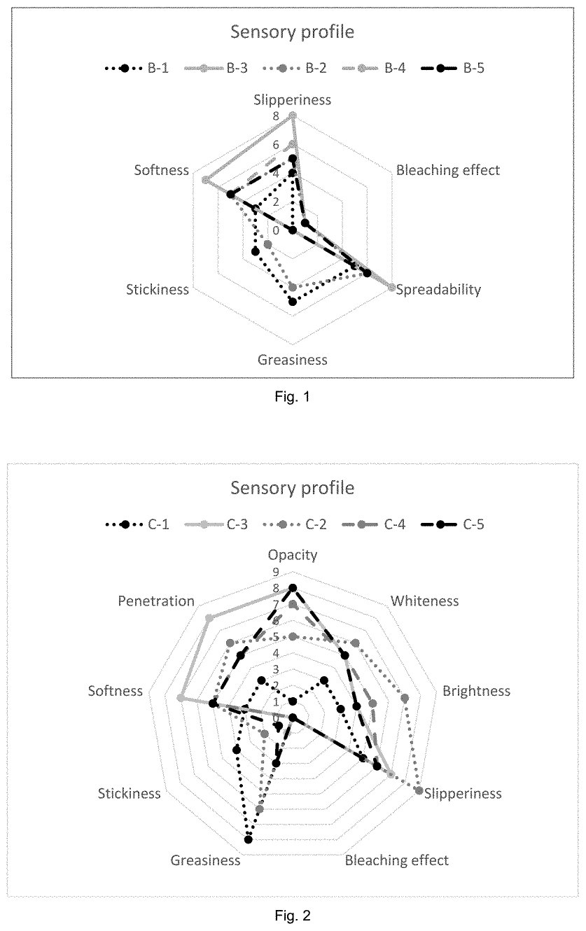

FIGS. 1 and 4 clearly demonstrate that the inventive skin cream composition B-3 enhances sensorial attributes like spreadbility, slipperiness and softness compared to the reference composition B-1 and the comparative composition B-2. Furthermore, FIG. 1 shows that the inventive composition B-3 reduces stickiness, greasiness and bleaching effect compared to the talc-containing comparative composition B-2 and the reference composition B-1. It can also be seen from FIGS. 1 and 4 that the spreadbility, slipperiness and softness of the inventive composition B-3 containing a combination of SRCC and NGCC is improved compared to the compositions B-4 and B-5, which contain only either surface-reacted calcium carbonate or ground calcium carbonate.

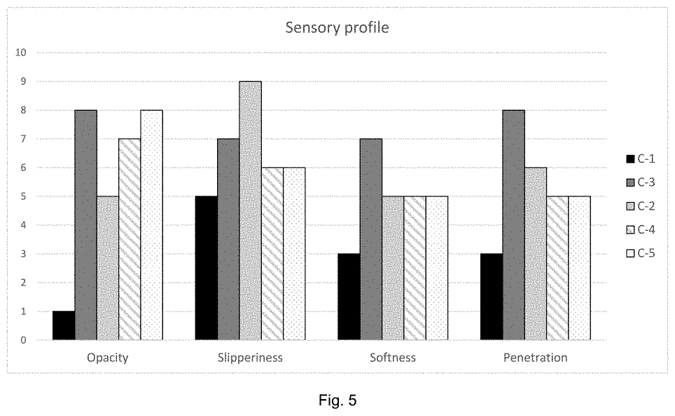

FIGS. 2 and 5 validate the positive effect of the inventive mineral blend in the deodorant stick (see inventive composition C-3) on softness and opacity compared to the comparative composition C-2 and the reference composition C-1. Furthermore, FIG. 2 shows that the inventive composition C-3 also reduces stickiness, greasiness and bleaching effect on the skin compared to talc-containing comparative composition C-2 and reference composition C-1. It can also be seen from FIGS. 2 and 5 that the penetration, slipperiness and softness of the inventive composition C-3 containing a combination of SRCC and NGCC is improved compared to the compositions C-4 and C-5, which contain only either surface-reacted calcium carbonate or ground calcium carbonate.

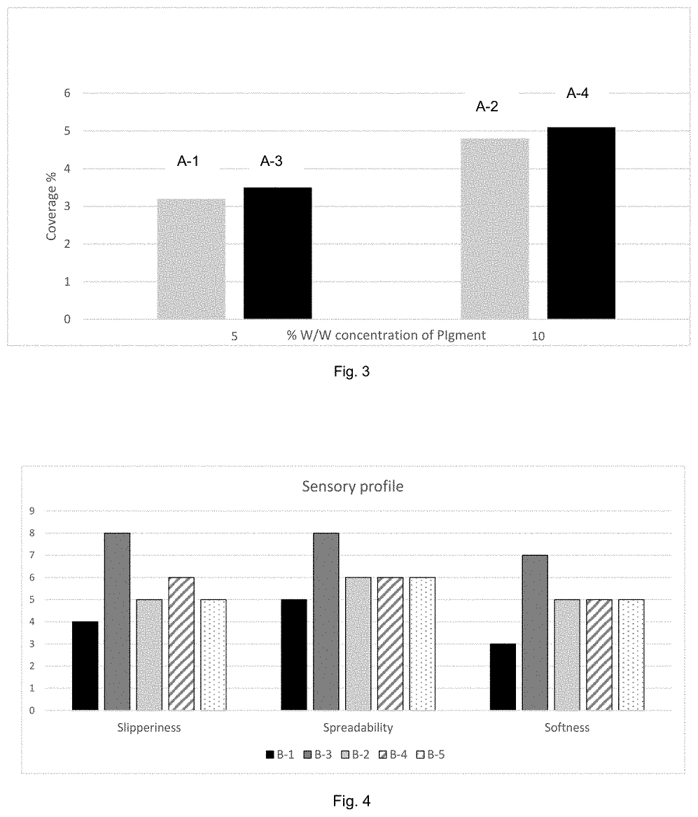

FIG. 3 clearly demonstrates the inventive base compositions A-3 and A-4 have a better coverage value than comparative compositions A-1 and A-2.

1. A method of using a mineral blend as a cosmetic agent for a wet cosmetic composition comprising the step of:
introducing the mineral blend into the wet cosmetic composition,
wherein the mineral blend comprises a first component being selected from natural ground calcium carbonate and/or precipitated calcium carbonate, and
a second component being a surface-reacted calcium carbonate, wherein the surface-reacted calcium carbonate is a reaction product of natural ground calcium carbonate or precipitated calcium carbonate with carbon dioxide and at least one H₃O⁺ ion donor, wherein the carbon dioxide is formed in situ by the at least one H₃O⁺ ion donor treatment and/or is supplied from an external source.

2. The method of using the mineral blend as set forth in claim 1, wherein the first component is present in an amount from 1 wt.-% to 99 wt.-%, preferably from 30 wt.-% to 99 wt.-%, more preferably from 50 wt.-% to 95 wt.-%, even more preferably from 60 wt.-% to 95 wt.-%, and most preferably from 70 wt.-% to 90 wt.-%, based on the total weight of the mineral blend, and
wherein the second component is present in an amount from 1 wt.-% to 99 wt.-%, preferably from 1 wt.-% to 70 wt.-%, more preferably from 5 wt.-% to 50 wt.-%, even more preferably from 5 wt.-% to 40 wt.-%, and most preferably from 10 wt.-% to 30 wt.-%, based on the total weight of the mineral blend.

3. The method of using the mineral blend as set forth in claim 1,
wherein the first component is a natural ground calcium carbonate selected from the group consisting of marble, chalk, limestone, and mixtures thereof, and/or
wherein the first component is a precipitated calcium carbonate selected from the group consisting of precipitated calcium carbonates having an aragonitic, vateritic or calcitic crystal form, and mixtures thereof.

4. The method of using the mineral blend as set forth in claim 1, wherein the at least one H₃O⁺ ion donor is selected from the group consisting of hydrochloric acid, sulphuric acid, sulphurous acid, phosphoric acid, citric acid, oxalic acid, an acidic salt, acetic acid, formic acid, and mixtures thereof, preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a cation selected from Li⁺, Na⁺ and/or K⁺, HPO₄²⁻, being at least partially neutralised by a cation selected from Li⁺, Na⁺, K⁺, Mg²⁺, and/or Ca²⁺, and mixtures thereof, more preferably the at least one H₃O⁺ ion donor is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

5. The method of using the mineral blend as set forth in claim 1,
wherein the first component has a volume median particle size d₅₀ from 0.1 to 50 μm, preferably from 0.5 to 40 μm, more preferably from 0.5 to 20 μm, even more preferably from 0.5 to 10 μm, and most preferably from 0.8 to 8 μm, and/or a specific surface area of from 0.5 m²/g to 30 m²/g, preferably from 1 m²/g to 20 m²/g, and more preferably from 2 m²/g to 15 m²/g, measured using nitrogen and the BET method, and/or
wherein the second component has a volume median particle size d₅₀ from 0.5 to 50 μm, preferably from 1 to 40 μm, more preferably from 1.2 to 30 μm, even more preferably from 1.5 to 15 μm, and most preferably from 3 to 10 μm, and/or a specific surface area of from 15 m²/g to 200 m²/g, preferably from 20 m²/g to 180 m²/g, more preferably from 25 m²/g to 160 m²/g, and most preferably from 30 m²/g to 90 m²/g, measured using nitrogen and the BET method.

6. The method of using the mineral blend as set forth in claim 1, wherein the surface-reacted calcium carbonate is associated with at least one active agent selected from pharmaceutically active agents, biologically active agents, disinfecting agents, preservatives, vitamins, flavouring agents, surfactants, oils, fragrances, and mixtures thereof.

7. The method of using the mineral blend as set forth in claim 1, wherein the wet cosmetic composition comprises a water content of at least 15 wt.-%, preferably at least 20 wt.-%, more preferably at least 25 wt.-%, and most preferably at least 30 wt.-%, based on the total weight of the wet cosmetic composition.

8. The method of using the mineral blend as set forth in claim 1, wherein the wet cosmetic composition is free of talc or a talc-containing material.

9. The method of using the mineral blend as set forth in claim 1, wherein the wet cosmetic preparation further comprises at least one additive selected from the group consisting of bleaching agents, thickeners, stabilizers, chelating agents, preserving agents, wetting agents, emulsifiers, emollients, fragrances, colorants, flavours, oils, skin tanning compounds, antioxidants, minerals, pigments, UV-A and/or UV-B filter, and mixtures thereof.

10. The method of using the mineral blend as set forth in claim 1, wherein the wet cosmetic preparation is in form of a paste, an ointment, a cream, a gel, a lotion, a solution, an emulsion, or a solid form.

11. The method of using the mineral blend as set forth in claim 1, wherein the wet cosmetic composition is a make-up product, preferably a lip make-up product, an eye make-up product, or a facial make-up product; a nail care product, a lip care product, a skin care product, a hair care product, a hair styling product, a hair colouring product, a deodorant, a hair remover, a make-up remover, a baby care product, a sun protection product, a tanner product, a feminine hygiene product, a bath product, a facial cleaning product, a hair cleaning product, a skin cleaning product, a soap product, or an oral care product.

12. The method of using the mineral blend as set forth in claim 1, wherein the mineral blend is introduced to the wet cosmetic composition in sufficient amounts for the wet cosmetic composition to absorb fluids, boost fragrance, decrease skin friction, modify the skin feel, and/or modify the skin appearance.

13. The method of using the mineral blend as set forth in claim 1, wherein the mineral blend is used as a replacement for talc or talc-containing materials.

14. A wet cosmetic composition comprising a mineral blend, wherein the mineral blend comprises
a first component being selected from natural ground calcium carbonate and/or precipitated calcium carbonate, and
a second component being a surface-reacted calcium carbonate, wherein the surface-reacted calcium carbonate is a reaction product of natural ground calcium carbonate or precipitated calcium carbonate with carbon dioxide and at least one H₃O⁺ ion donor, wherein the carbon dioxide is formed in situ by the at least one H₃O⁺ ion donor treatment and/or is supplied from an external source, and
the wet cosmetic composition is a make-up product, preferably a lip make-up product, an eye make-up product, or a facial make-up product; a nail care product, a lip care product, a skin care product, a hair care product, a hair styling product, a hair colouring product, a deodorant, a hair remover, a make-up remover, a baby care product, a sun protection product, a tanner product, a feminine hygiene product, a bath product, a facial cleaning product, a hair cleaning product, a skin cleaning product, a soap product, or an oral care product.

15. The wet cosmetic composition of claim 14, wherein the wet cosmetic composition comprises the mineral blend in an amount from 0.1 to 50 wt.-%, based on the total weight of the wet cosmetic composition, preferably from 0.5 to 20 wt. %, more preferably from 1 to 10 wt.-%, and most preferably from 3 to 6 wt.-%.

16. The wet cosmetic composition of claim 14, wherein the first component is present in an amount from 70 wt.-% to 90 wt.-% based on the total weight of the mineral blend, and wherein the second component is present in an amount from 10 wt.-% to 30 wt.-%, based on the total weight of the mineral blend.

17. The wet cosmetic composition of claim 14, wherein the at least one H₃O⁺ ion donor is selected from the group consisting of hydrochloric acid, sulphuric acid, sulphurous acid, phosphoric acid, oxalic acid, and mixtures thereof.

18. The wet cosmetic composition of claim 14,
wherein the first component has a volume median particle size d₅₀ from 2 m²/g to 15 m²/g, measured using nitrogen and the BET method, and/or
wherein the second component has a volume median particle size d₅₀ from 30 m²/g to 90 m²/g, measured using nitrogen and the BET method.

19. The wet cosmetic composition of claim 14, wherein the surface-reacted calcium carbonate is associated with at least one active agent selected from pharmaceutically active agents, biologically active agents, disinfecting agents, preservatives, vitamins, flavouring agents, surfactants, oils, fragrances, and mixtures thereof.

20. The wet cosmetic composition of claim 14, wherein the wet cosmetic preparation further comprises at least one additive selected from the group consisting of bleaching agents, thickeners, stabilizers, chelating agents, preserving agents, wetting agents, emulsifiers, emollients, fragrances, colorants, flavours, oils, skin tanning compounds, antioxidants, minerals, pigments, UV-A and/or UV-B filter, and mixtures thereof.
